(19) United States
(12) Patent Application Publication
(10) Pub. No.: US 2004/0259942 A1
(43) Pub. Date: Dec. 23, 2004

(54) Modulation of stat activity

(76) Inventors: Peter Shaw, Wollaton Nottingham (GB); David Pritchard, Nottingham (GB); Li Li, Beeston Nottingham (GB)

Correspondence Address: Fitzpatrick Cella Harper & Scinto, 30 Rockfeller Plaza, New York, NY, 10112-3800, US

(21) Appl. No.: 10/490,339

(22) PCT Filed: Sep. 17, 2002
(86) PCT No.: PCT/GB02/04232

(30) Foreign Application Priority Data
Sep. 22, 2001 (GB) 0122914.5

(51) Int. Cl. A61K 31/195

(52) U.S. Cl. 514/473; 514/561

(57) ABSTRACT

The use of a compound selected from a range of compounds including quorum sensing molecules, N-acyl homo serine lactones, N-(3-oxododecanoyl)-L-homoserine lactone, inhibitors to modulate STAT activity for the treatment of a range of diseases including cancer, breast cancer, obesity, lipid metabolism disorders, immune disease, immune deficiency or immune disorders. The range of compounds also include compounds of formula (I) in which R is an acyl group of formula (II).

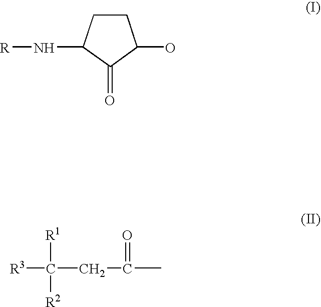

### FIELD OF THE INVENTION

The invention relates to the modulation of STAT activity. The invention also relates to compounds preferably (though not exclusively) quorum sensing molecules such as those produced by Pseudomonas aeruginosa for inhibiting STAT activation.

### REVIEW OF THE ART KNOWN TO THE APPLICANT(S)

STATS (Signal Transducers and Activators of Transcription) are evolutionarily conserved molecules (proteins) identified in the genomes of mammals, flies, worms and even the slime mould Dictostelium. Inactive STAT proteins are cytoplasmic or associated with membrane growth factor and cytokine receptors. Ligand binding to these receptors causes the tyrosine phosphorylation of associated STATS, their homo- or heterodimerisation and translocation to the cell nucleus, where they interact with promotor elements to activate target gene expression.

Biological functions of mammalian STAT proteins have been revealed by gene targeting experiments in mice. STAT1 knockout mice show defective macrophage function and sensitivity to viral infection, while the absence of STAT5a and STAT5b causes defects in T cell growth. Notably, mice lacking STAT4 are defective in Th1 responses and STAT6-deficient mice are defective in Th2 responses. Deletion of the gene for STAT3 results in early embryonal lethality. This is possibly due to the singular role of STAT3 in the proliferation of several cell types. Conditional deletions of STAT3, however, demonstrate a requirement in T cells for IL-2 and IL-6-induced proliferation, in macrophages to counteract chronic inflammation and in keratinocytes for wound healing. Thus, consistent with findings that link the invertebrate STAT protein to immune function, a common theme in STAT knock-out mice is the disruption of aspects of immune function.

### SUMMARY OF THE INVENTION

In its broad concept the invention provides an autocrine/paracrine signalling pathway which is associated with cell growth/proliferation, modifications to which can be used to alter cell growth and/or cell proliferation.

In one aspect the invention provides an autocrine/paracrine signalling pathway which activates STAT wherein the pathway requires JAK activity and does not require Erb1 activity and is not induced by EGF.

In a further aspect the invention provides a process wherein STAT dimmers accumulate in the cytoplasm wherein the process does not require Erb1 activity or JAK activity.

In a further aspect the invention enables the modulation of any one of these pathways or processes to alter the amount of activated STAT.

In yet a further aspect the invention encompasses the use of a compound selected from:—JAK, ErbB1, EGF, ErbB1 inhibitors, EGF inhibitors, STAT inhibitors, interleukin-13 (IL-13), IL-13E13K (IL-13 in which the Glu at position 13 is substituted by a Lys residue), sulpher methoxyzol, ubiquitin E3 ligase, serine phosphatase, tyrosine phosphotase, SOCs, Pias proteins (protein inhibitors of activated STAT), STAT1 inhibitors, STAT2 inhibitors, STAT3 inhibitors, STAT4 inhibitors, STAT5A inhibitors, STAT5B inhibitors, STAT6 inhibitors, JAK inhibitors, AG 490, α-amanitin, transcription inhibitors, quorum sensingmolecules, N-acyl homoserine lactones, N-(3-oxododecanoyl)-L-homoserine lactone, oxygen radical scavengers, N-acetyl Cysteine (NAC), diphenylene iodonium chloride (DPI), inhibitors of COX1, inhibitors of COX2, aspirin, ketorolac, indomethacin, or panCOX inhibitors to modulate STAT activity for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

In another aspect the invention relates to the use of a compound of the formula I

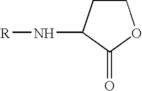

in which R ia an acyl group of the formula II

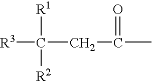

wherein one of R¹ and R² is H and the other is selected from OR⁴, SR⁴ and NHR⁴, wherein R⁴ is H or 1-6C alkyl, or R¹ and R² together with the carbon atom to which they are joined form a keto group and R³ is a straight or branched chain saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of the R⁵ and R⁶ is selected from H and 1-6C alkyl or R⁵ and R⁶ together with the N atom from a morpholino or piperazino group or any enantiomer thereof with the proviso that R is not a 3-oxododecanoyl group to modulate STAT activity for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

In a further aspect the invention relates to any one of these uses wherein the R group is selected from

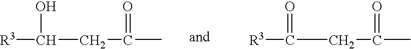

wherein R³ is as defined above.

In another aspect the invention relates to any of these uses wherein the group R³ is an 8-11C straight or branched chain alkyl group optionally substituted by a substituent selected from bromo, carboxy and methoxycarbonyl.

In a further aspect the invention relates to any one of these uses wherein the R³ group is such that the group R in formula I is selected from;

3-oxoundecanoyl;

11-bromo-3-oxoundecanoyl;

10-methyl-3-oxoundecanoyl;

6-methyl-3-oxoundecanoyl;

3-hydroxydodecanoyl;

12-bromo-3-oxododecanoyl;

3-oxotridecanoyl;

13-bromo-3-oxotridecanoyl;

3-hydroxytetradecanoyl;

3-oxotetradecanoyl;

14-bromo-3-oxotradecanoyl; and

13-methoxycarbonyl-3-oxotridecanoyl.

In a further aspect the invention relates to any one of these uses wherein the R³ is an 8-11 straight or branched chain alkenyl group optionally substituted by a substituent selected from bromo, carboxy and methoxycarbonyl.

In a further aspect the invention relates to any one of these uses wherein the R³ group is such that the group R in formula I is selected from;

3-oxo-12-tridecenoyl;

3-oxo-7-tridecenoyl;

3-hydroxy-7-tetradecenoyl;

3-oxo-9-tetradecenoyl;

3-hydroxy-9-tetradecenoyl;

3-oxo-10-tetradecenoyl;

3-hydroxy-10-tetradecenoyl;

3-oxo-11-tetradecenoyl;

3-hydroxy-11-tetradecenoyl;

3-oxo-13-tetradecenoyl; and

3-hydroxy-13-tetradecenoyl.

In another aspect the invention relates to the use of JAK, ErbB1, EGF, ErbB1 inhibitors, EGF inhibitors, STAT inhibitors, interleukin-13 (IL-13), IL-13E13K (IL-13 in which the Glu at position 13 is substituted by a Lys residue), sulpher methoxyzol, ubiquitin E3 ligase, serine phosphatase, tyrosine phosphotase, SOCs, Pias proteins (protein inhibitors of activated STAT), STAT1 inhibitors, STAT2 inhibitors, STAT3 inhibitors, STAT4 inhibitors, STAT5A inhibitors, STAT5B inhibitors, STAT6 inhibitors, JAK inhibitors, AG 490, α-amanitin, transcription inhibitors, quorum sensing molecules, N-acyl homoserine lactones, N-(3-oxododecanoyl)-L-homoserine lactone, oxygen radical scavengers, N-acetyl Cysteine (NAC), diphenylene iodonium chloride (DPI), inhibitors of COX1, inhibitors of COX2, aspirin, ketorolac, indomethacin, or panCOX inhibitors, for the preparation of a medicament for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

In a further aspect the invention relates to the use of a compound of the formula I

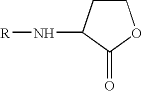

in which R is an acyl group of the formula II

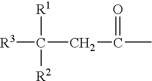

wherein one of R¹ and R² is H and the other is selected from OR⁴, SR⁴ and NHR⁴ wherein R⁴ is H or 1-6C alkyl, or R¹ and R² together with the carbon atom to which they are joined form a keto group and R³ is a straight or branched chain saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of the R⁵ and R⁶ is selected from H and 1-6C alkyl or R⁵ and R⁶ together with the N atom from a morpholino or piperazino group or any enantiomer thereof with the proviso that R is not a 3-oxododecanoyl group for the preparation of a medicament for the treatment of treatment of cancer, breast cancer, multiple myeloma, head and neck cancers, leukaemia, HTLV-1-dependent leukemia, large granular lymphocte (LGL) leukemia, erythroleukemia, acute lymphocytic leukemia (ALL), chronic lymphocytic leukemia (CLL), acute myelogenous leukemia (AML), chronic myelogenous leukemia (CML), megakaryotic leukemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (Tcell) lymphoma, cutaneous T cell lymphoma, obesity, lipid metabolism disorders, immune disease, immune deficiency or immune disorders.

### DESCRIPTION OF THE DRAWINGS

The invention will be described by way of example with reference to the accompanying drawings in which:

FIG. 1 is a diagrammatic representation of the autocrine/paracrine pathway which activates STAT3.

FIG. 2 shows:

ErbB and STAT protein expression in BC cell lines. Lysates were prepared from BT20 (lane 1), MCF-7 (lane 2), T47D (lane 3), MDA-MB-231 (lane 4), MDA-MB-468 (lane 5) and BR293 (lane 6) cells. For ErbB1, STAT1 and STAT3 200 μg protein from each cell lysate were used for Erb2 and Erb3 400 μg of protein was used and separated by SDS-PAGE, transferred to PVDF membranes and probed with anti-ErbB (upper panel) or anti-STAT (lower panel) antibodies as indicated. One set of lysates was used throughout.

FIG. 3 shows: Tyrosine phospborylation of ErbB proteins in BC cell lines. Lysates were prepared (see Materials and Methods) from BT20 (lanes 1, 2), MCF-7 (lanes 3, 4), T47D (lanes 5, 6) MDA-MB-231 (lanes 7, 8), MDA-MB-468 (lanes 9, 10) and BR293 (lanes 111, 12) cells that had been serum-starved (−) or starved and treated with EGF (5 nM) for 15 min (+). ErbB proteins were collected as immune complexes, separated by SDS-PAGE, transferred to PVDF membrane and probed first with an anti-phosphotyrosine antibody and subsequently with the corresponding anti-ErbB antibody, as indicated. ND indicates that the protein is not expressed at detectable levels by the cell line (see FIG. 2). Numbers below each panel show the level of tyrosine phosphorylation, quantified with Image Quant software (Fuji) and expressed as the ratio αPY/αErbB, whereby the unstimulated value for each protein in each cell line is set as 1. The results shown are compiled from several experiments in which ErbB proteins from each cell line were analysed at least three times with similar results.

FIG. 4 shows:

Tyrosine phosphorylation of STAT1 and STAT3 proteins in BC cell lines. Lysates were prepared from BT20 (lanes 1 and 2), MCF-7 (lanes 3 and 4) T47D (lanes 5 and 6), MDA-MB-231 (lanes 7 and 8), MDA-MB-468 (lanes 9 and 10) and BR93 cells (lanes 11 and 12) that had been serum-starved (−) or starved and treated with EGF (5 nM) for 15 min (+). Proteins (200 μg) were separated by SDS-PAGE, transferred to PVDF membrane and probed first with an anti-phosph-STAT1 or anti-phospho-STAT3 antibody and subsequently with the corresponding anti-STAT antibody as indicated.

FIG. 5 shows:

EGF-induced formation of DNA complexes by STAT 1 proteins in BC cells. Extracts were prepared from MDA-MB-468 (lanes 1-6), BT20 (lanes 7-12) and BR293 cells (lanes 13-18) that had been serum-starved (−) or starved and treated with EGF (5 nM) for 15 min (+). Equal amounts (25 μg protein) of each extract were incubated alone (lanes 1, 4, 7, 10, 13 and 16) or with antibodies specific for STAT1 (lanes, 2, 5, 8, 11, 14 and 17) or STAT3 (lanes 6, 9, 12, 15 and 18) and aradiolabelled oligonucleotide duplex corresponding to the M67 sequence derived from the c-fos SIE. STAT1 homodimers (1:1), STAT3 homodimers (3:3), heterogeneous STAT3 complexes (3H) and supershifted STAT3 complexes (3SS) are indicated. In subsequent figures only the upper parts of the EMSA gels are shown.

FIG. 6 shows:

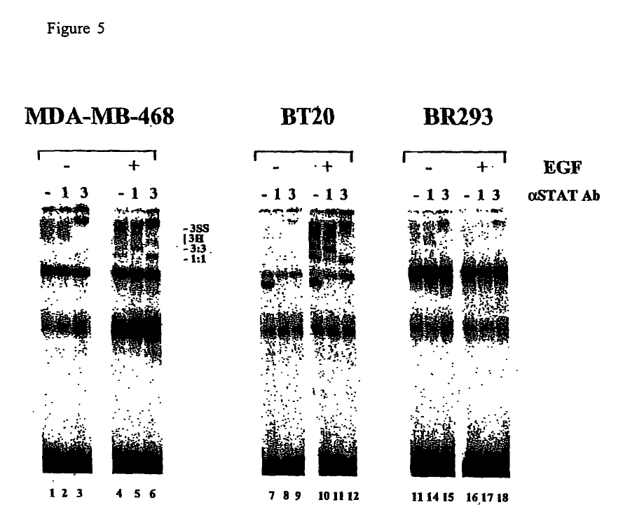

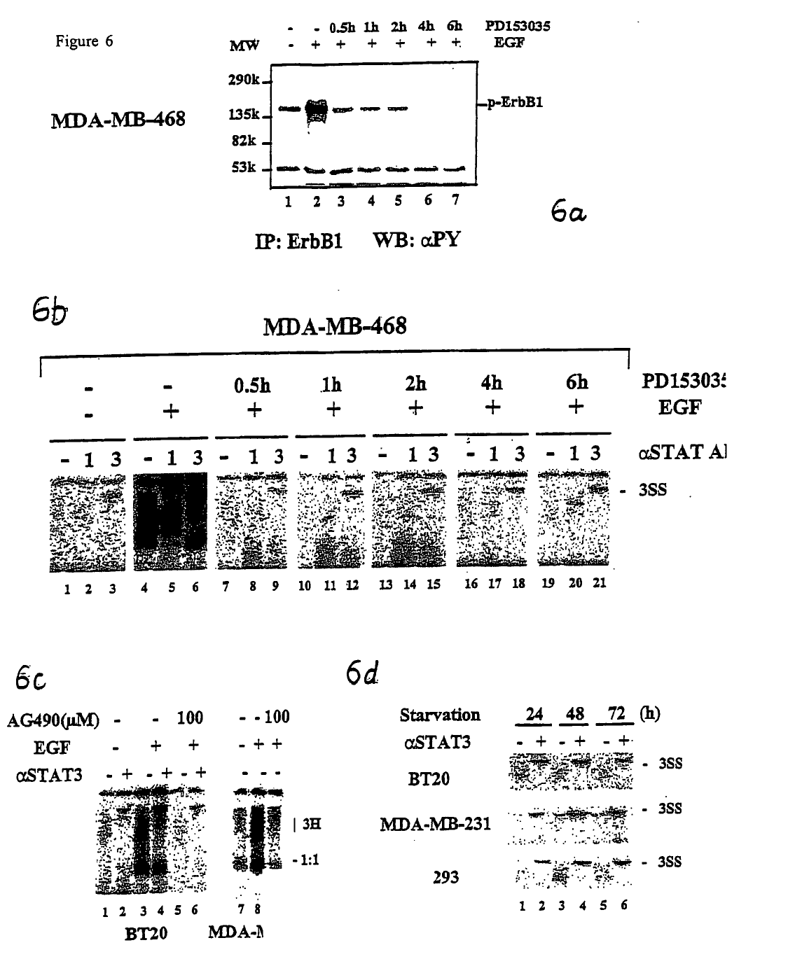

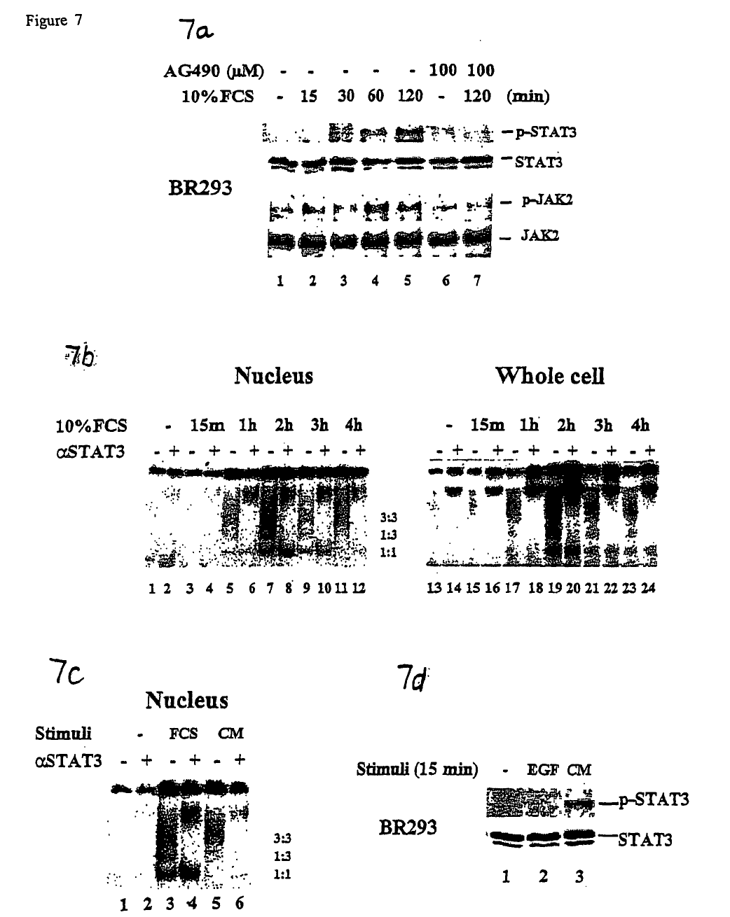

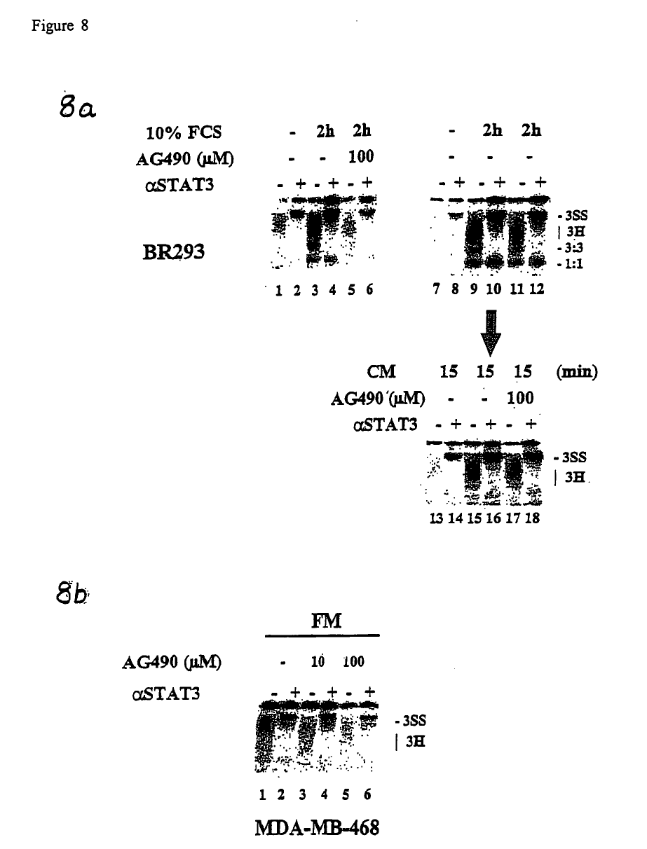

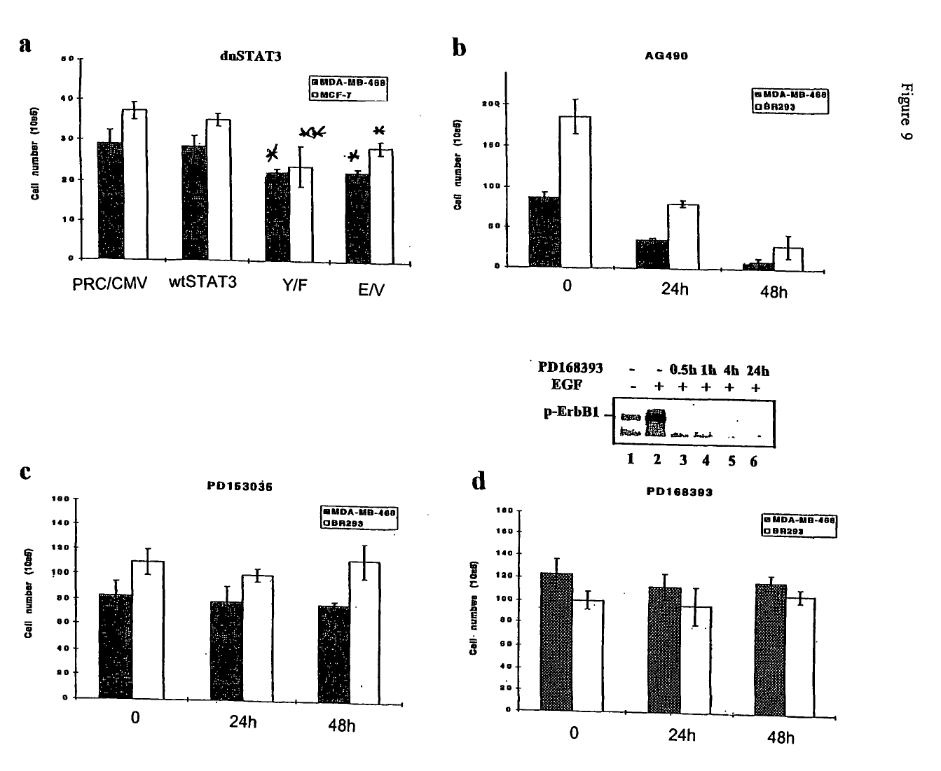

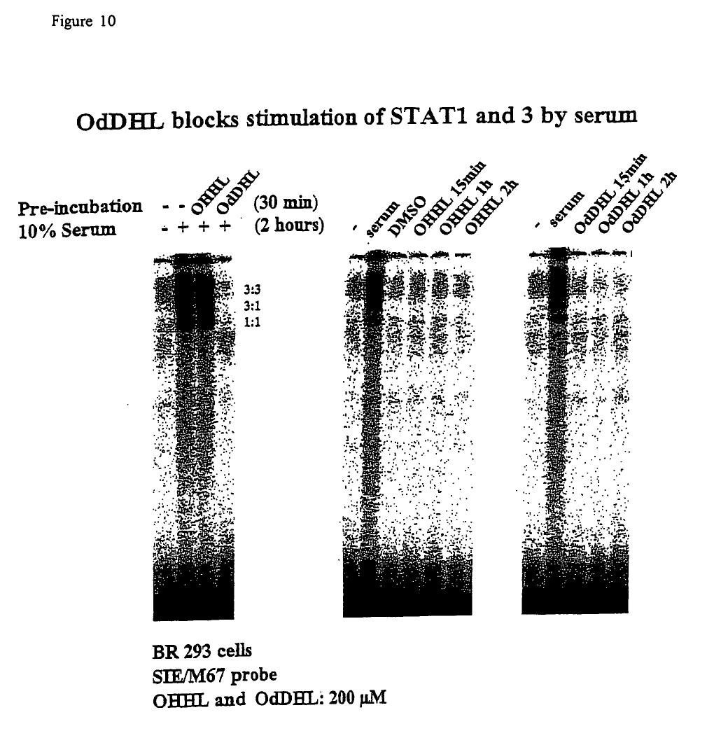

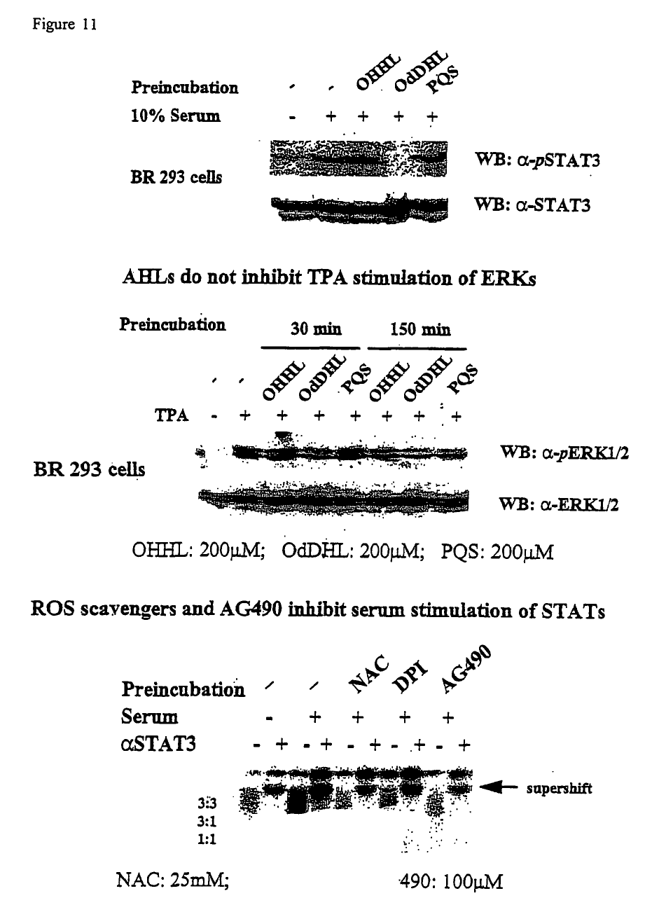

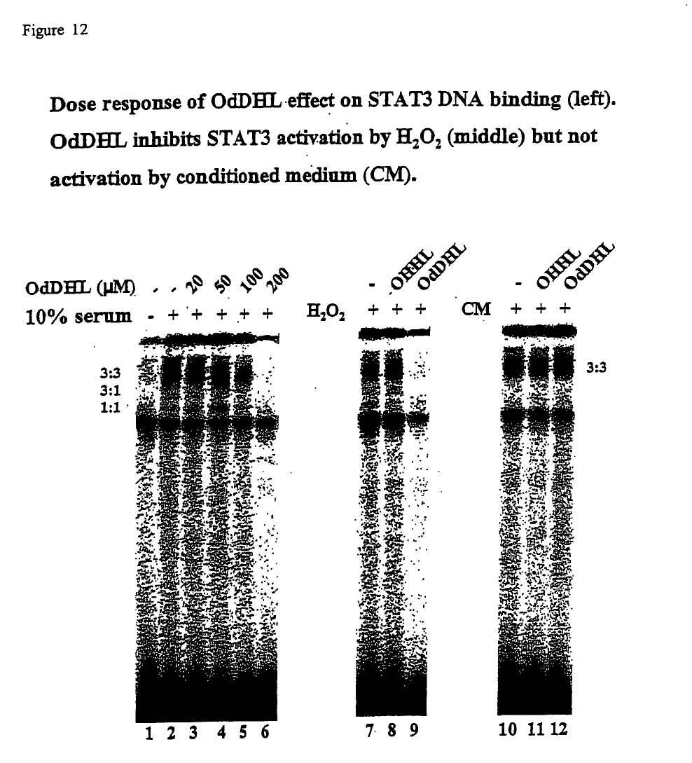

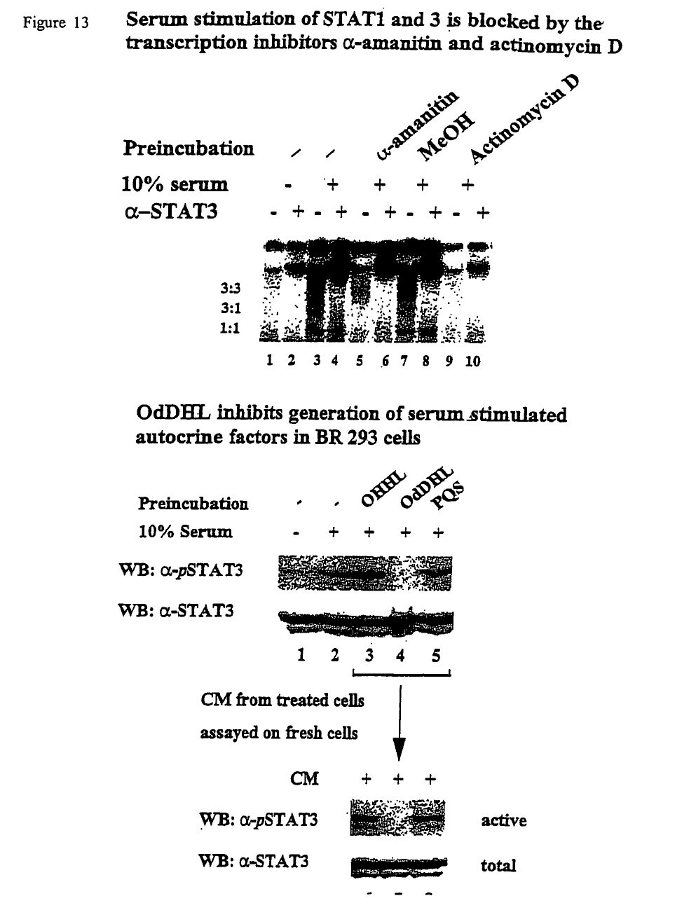

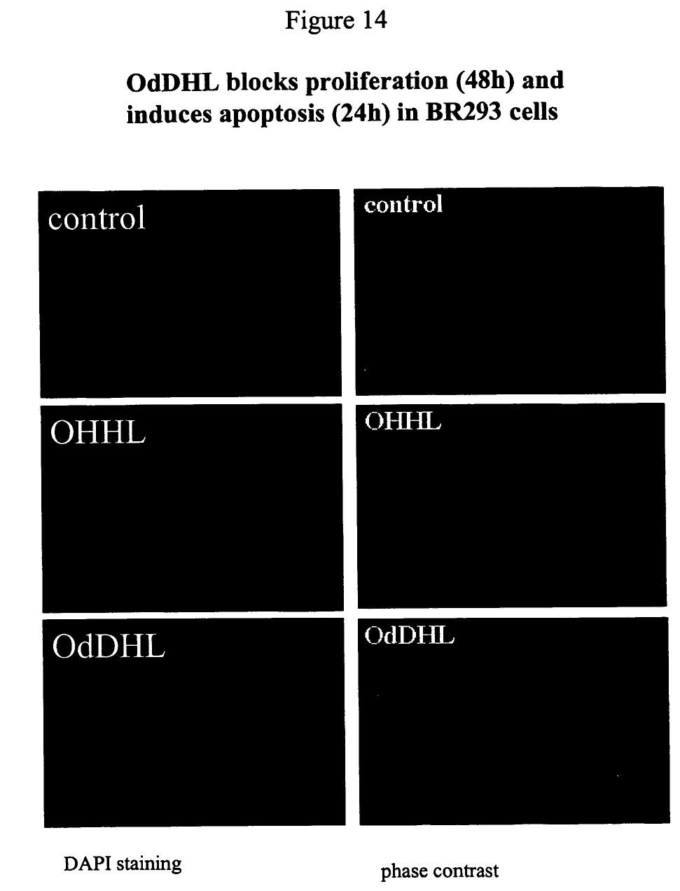

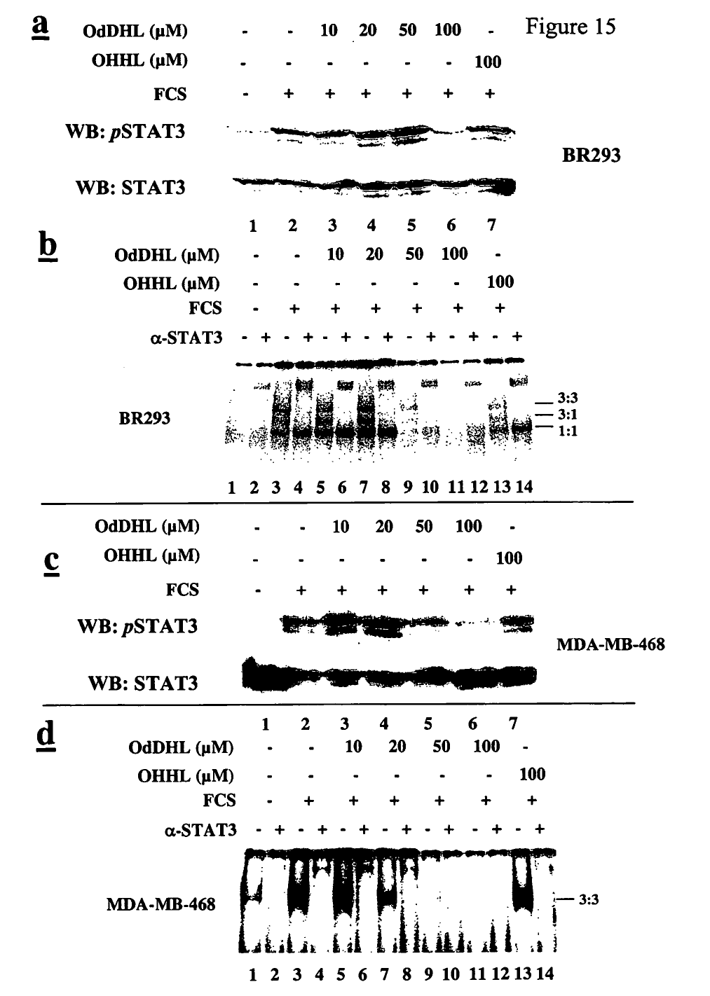

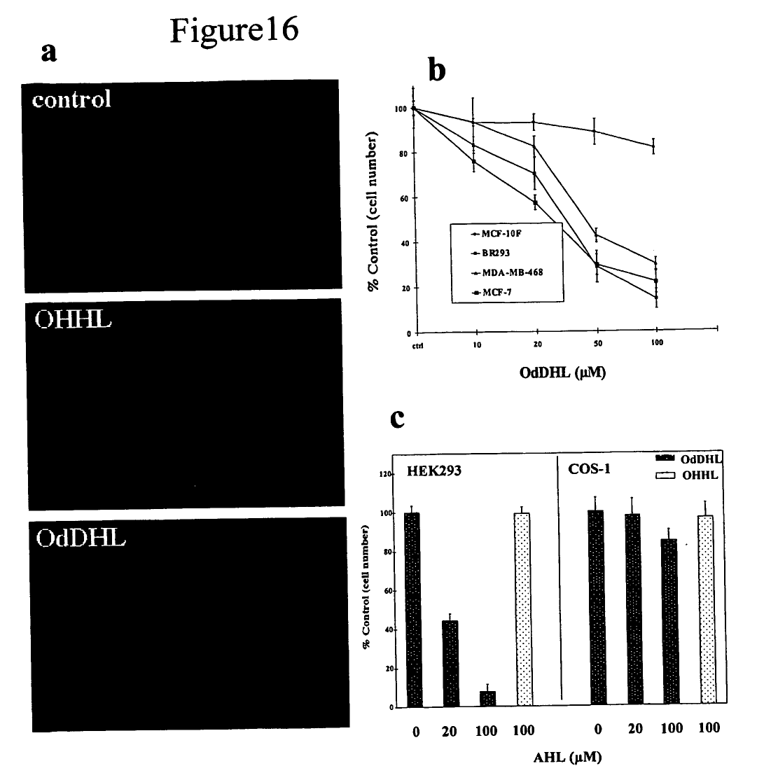

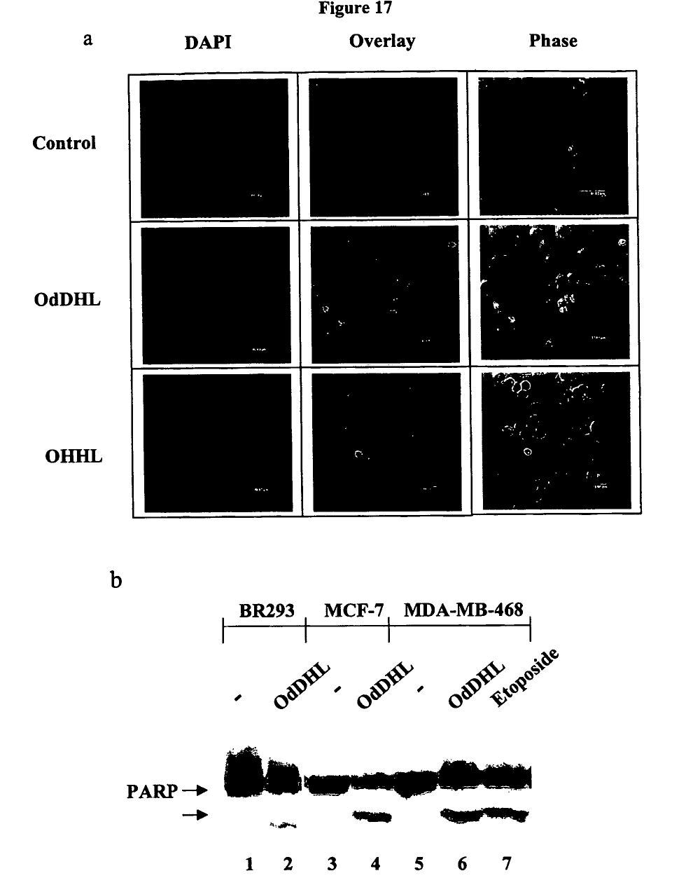

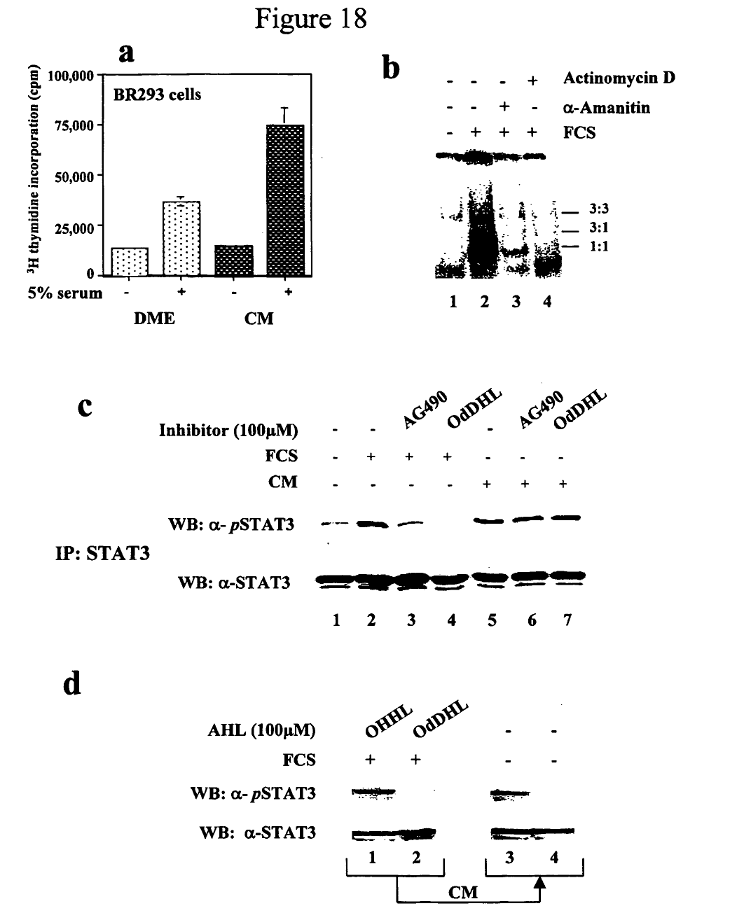

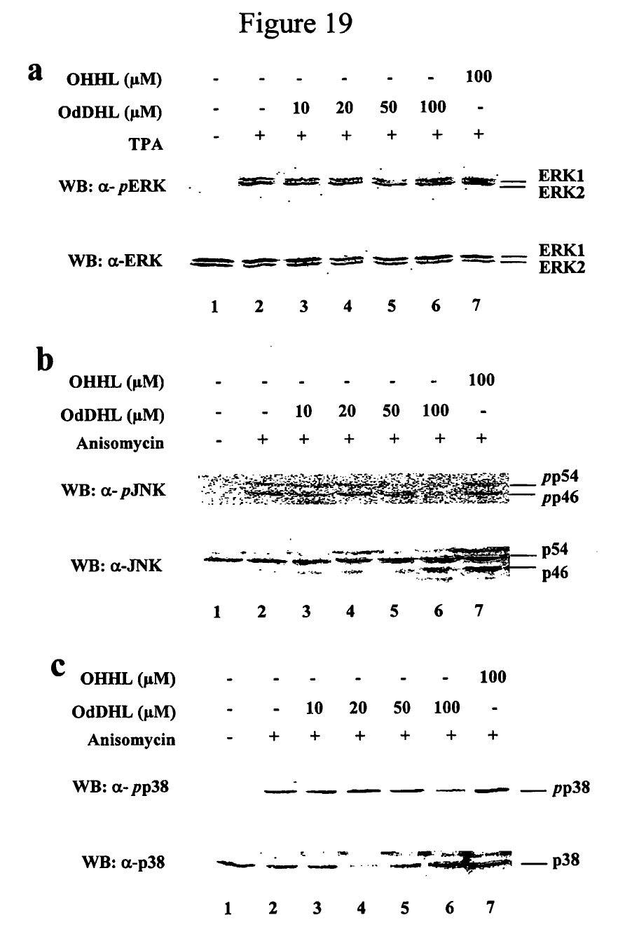

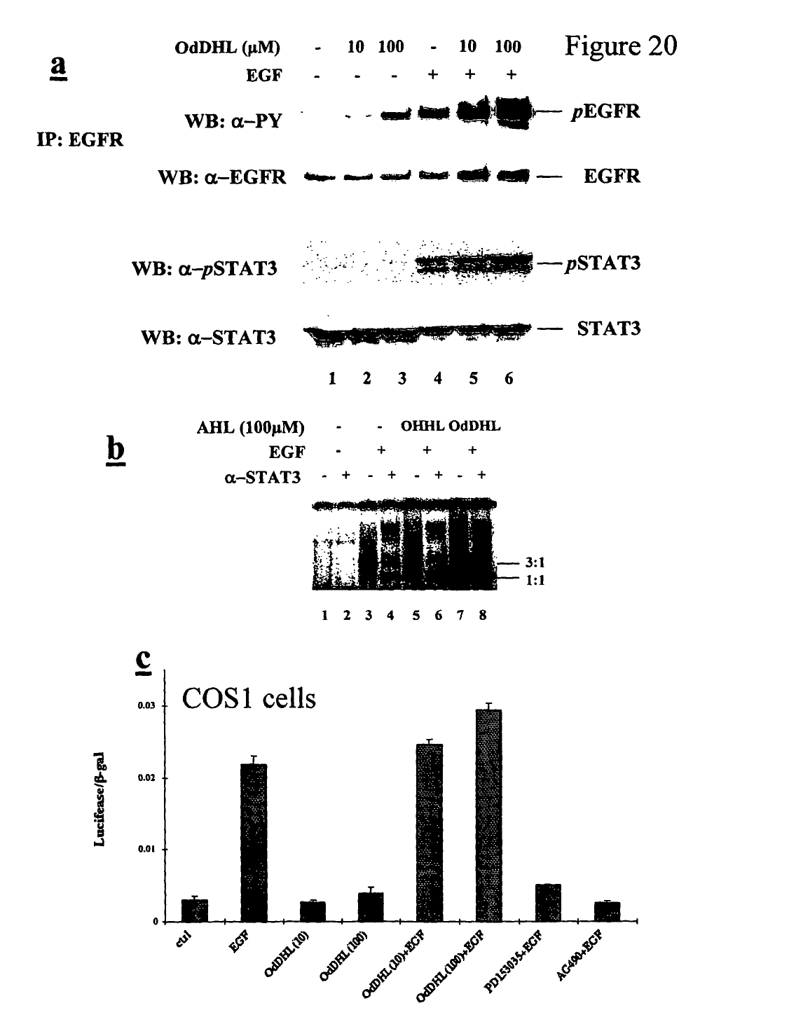

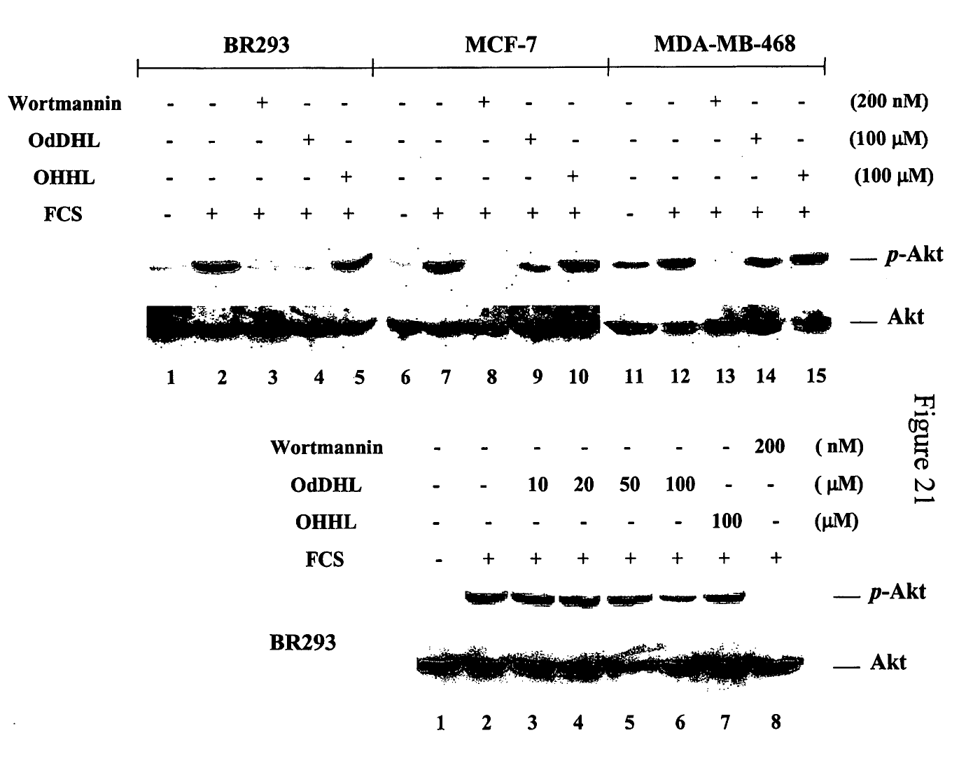

Inhibition of EGF-induced phosphorylation of ErbB1 and DNA binding of STAT proteins. (a) Serum-starved MDA-MB-468 cells were pre-treated with PD153035 (100 nM) for the times indicated and then treated with EGF (5 nM) for 15 min (+). Lysates were prepared, from which ErbB1 proteins were collected as immune complexes, separated by SDS-PAGE, transferred to PVDF membrane and probed with an anti-phosphotyrosine antibody as indicated. (b) Equal amounts (25 μg protein) of each lysate were incubated alone (lanes 1, 4, 7, 10, 13, 16 and 19) or with antibodies specific for STAT1 (lanes 2, 5, 8, 11, 14, 17 and 20) or STAT3 (lanes 3, 6, 9, 12, 15, 18 and 21) and a radiolabelled oligonucleotideduplex corresponding to the M67 sequence derived from the c-fos SIE. In this and subsequent figures only the upper parts of the EMSA gels are shown. (c) BT20 (lanes 1-6) and MDA-MB-468 cells (lanes 7-9) were serum-starved (−), treated with EGF (5 nM) for 15 min (+) or pre-treated with AG490 (100 μM) for 30 min and then treated with EGF (5 nM) for 15 min (+). DNA binding by STAT proteins was analysed as described in the legend to FIG. 5. The STAT1 homodimer is indicated (1:1). (d) BT20 (upper), MDA-MB-231 (middle) and BR293 cells (lower panel) were incubated in serum-free medium for 24 (lanes 1, 2), 48 (lanes 3, 4) or 72 hours (lanes 5, 6). Cell extracts were prepared and equal amounts of each were incubated directly with the M67 DNA probe (lanes 1, 3, 5) or after pre-incubation with an anti-STAT3 antibody (lanes 2, 4, 6). STAT1 homodimers (1:1) and supershifted STAT3 complexes (3SS) are indicated.

FIG. 7 shows:

Delayed Activation of STAT3 Involves Autocrine/Paracrine Signalling.

(a) Extracts were prepared from serum-starved BR293 cells (lanes 1 and 6) or starved cells stimulated directly with 10% FCS for the times indicated (lanes 2-5), or after pre-treatment with AG490. STAT3 and JAK2 proteins were collected as immune complexes, separated by SDS-PAGE, transferred to PVDF membrane and probed first with an anti-phosphotyrosine antibody and subsequently with

anti-STAT3 or anti-JAK2 antibodies as indicated. (b) Serum-starved BR293 cells were stimulated with 10% FCS for the times indicated and activation of STAT DNA-binding was assayed with the M67 DNA probe in nuclear (left panel) and whole cell extracts (right panel). (c) BR 293 cells were serum-starved (lanes 1 and 2), or serum-starved and treated with 10% FCS for 2 hours (lanes 3 and 4).

Alternatively, after 2 hours in 10% FCS, cells were washed and incubated in serum-free medium for a further 4 hours, whereupon conditioned medium from the cells was transferred to fresh serum-starved BR293 cells, which were harvested after 15 minutes (lanes 5 and 6). STAT DNA-binding activity in nuclear extracts was assayed with the M67 DNA probe. (d) Extracts were prepared from serum-starved BR293 cells (lane 1) and cells stimulated with EGF (lane 2) or conditioned medium (lane 3) for 15 min. STAT3 proteins were collected as immune complexes, separated by SDS-PAGE, transferred to PVDF membrane and probed first with an anti-phosphotyrosine antibody and subsequently with an anti-STAT3 antibody as indicated.

FIG. 8 shows:

Inhibition of serum-induced STAT DNA binding. (a) BR293 cells were left untreated (lanes 1, 2) or treated with 10% FCS for 2 hours in the absence (lanes 3, 4) or presence of 100 μM AG490 (lanes 5, 6). In addition, conditioned medium from serum-starved cells (lanes 7, 8) or from cells incubated in 10% FCS for 2 h (lanes 9-12) was transferred for 15 minutes to fresh serum-starved cells (lanes 13-16) or starved cells pre-treated with 1001M AG490 (lanes 17, 18). Extracts were prepared from all the cells and DNA binding by STAT proteins was analysed as described in the legends to FIG. 5. (b) MDA-MD-468 cells growing in full medium (FM) were treated with AG490 for 24 hours at the concentrations indicated. DNA binding by STAT proteins was analysed as described in the legend to FIG. 5. STAT1 homodimers (1:1), STAT3 homodimers (3:3), heterogeneous STAT3 complexes (3H) and supershifted STAT3 complexes (3SS) are indicated.

FIG. 9 shows:

Inhibition of BC cell growth. (a) Equal numbers of MDA-MB-468 and MCF-7 cells, transfected with a control vector or expression vectors for wild type of dominant-inhibitory versions of STAT3 (Y/F and EN: see Materials and Methods) were plated in full medium and cultured for 96 hours. Cells were then harvested and counted. Data are expressed as means±S.D. Significant differences between groups were determined by Student's t-test. P values <0.05 (*) are considered significant. ** indicates P values <0.01. (b) Equal numbers (1×10⁶) of MDA-MB468 and BR293 cells were plated in full medium and cultured for 60 hours. Ag490 (1001M) was added 48 and 24 hours prior to counting or omitted entirely. (c) As in (b) except that 1×10⁶ MDA-MB-468 cells but only 5×10⁵ BR293 cells were plated and the ErbB1 inhibitor PD153035 (100 nM) was used. (d) As in (c)except that the irreversible ErbB1 inhibitor PD 168393 (2 μM) was used. Inset shows ErbB1 tyrosine phosphorylation levels in MDA-MB468 cells treated with PD 168393 (2 μM) over 24 hours. Results are expressed as cell number after 48 hours growth, whereby error bars show the standard error from triplicate points.

FIG. 10 shows: The effect of OdDHL on the serum-induced accumulation of STAT1 and STAT3.

FIG. 11 upper panel shows: The effect of OdDHL on serum-induced phosphorylation of STAT3; middle panel the effect of N-acyl homoserine lactones (AHL) on TPA stimulation of ERKs; lower panel the effect of reactive oxygen (ROS) scavengers, and the JAK inhibitor AG490 on serum stimulation of STATs.

FIG. 12 shows: Dose-response of OdDHL on DNA binding by STAT1+STAT3, inhibition of ROS-induced DNA binding by STAT1+STAT3 but not CM-induced DNA binding by STAT3.

FIG. 13 upper panel shows: The effect of transcription inhibitors on serum-mediated stimulation of STAT1+STAT3; lower panel shows that the conditioned medium from serum+OdDHL-treated cells lacks the autocrine factor required to induce STAT3 phosphorylation.

FIG. 14 shows: The effect of OdDHL on breast cancer cell proliferation (right hand panels) and apoptosis (left hand panels).

FIG. 15 shows:

Serum-stimulation of STAT3 is inhibited by OdDHL. (a) BR293 cells were serum-starved (lane 1) or starved and stimulated with 10% FCS for 2 hours alone (lane 2) or in the presence of increasing concentrations of OdDHL (lanes 3-6) or 100 μM OHHL (lane 7). Cell lysates were prepared and analysed by Western blotting for STAT3 phosphorylation with an antibody specific for STAT3 phosphorylated on Y705 (upper panel) and for STAT3 content with an antibody for STAT3 (lower panel). (b) From BR293 cells treated as in (a) nuclear extracts were prepared and analysed for STAT1 and STAT3 DNA-binding activity by EMSA with a radio-labelled probe corresponding to the M67 SIE. The inclusion of an anti-STAT3 antibody in duplicate binding reactions (even lanes) identifies STAT3-containing homo-(3:3) and heterodimers (3:1), as indicated to the right of the panel. (c) MDA-MB-468 cells were treated and analysed exactly as described for BR293 cells in (a). (d) MDA-MB-468 cells were treated and analysed exactly as described for BR293 cells in (b).

FIG. 16 shows:

OdDHL inhibits proliferation of BC cells (a) BR293 cells (1×10⁶) were cultured in full medium in the presence of DMSO control (upper panel), 100 μM OHHL (middle panel) or 100 μM OdDHL (lower panel). After 48 hours cells were photographed. (b) Equal numbers of MCF-10F, MCF-7, MDA-MB-468 (1.5×10⁶) and BR293 cells (1×10⁶) were cultured in full medium alone or in the presence of various concentrations of OdDHL. After 48 hours cells were harvested and counted in a haemocytometer. Values are given as means of triplicate points, whereby error bars indicate standard errors. (c) Equal numbers of HEK293 and COS-1 cells (1×10⁶) were cultured in full medium alone or in the presence of various concentrations of OdDHL or 1001M OHHL. After 48 hours cells were harvested and counted in a haemocytometer. Values are given as means of triplicate points, whereby error bars indicate standard errors.

FIG. 17 shows:

OdDHL induces apoptosis of BC cells (a) MCF-7 cells were cultured in full medium alone or in the presence of 1001M OdDHL or 100 μM OHHL. After 18 hours cells were fixed, stained with DAPI and examined by confocal microscopy. Left-hand panels show DAPI, right-hand panels show the corresponding phase contrast and middle panels the image overlays. (b) MDA-MB-468 cells were cultured in full medium alone or in the presence of 100 μM OdDHL or 100 μM OHHL. After 18 hours cells were fixed, stained and examined as in (a). (c) BR293, MCF-7 and MDA-MD-468 cells were cultured in full medium alone (lanes 1, 3, 5) or in the presence of 100 μM OdDHL (lanes 2, 4, 6) or 400 μM etoposide (lane 7). After 18 hours cells were lysed and analysed for PARP cleavage by Western blotting with an anti-PARP antibody. Full length PARP is labelled and the lower arrow indicates the major caspase cleavage fragment.

FIG. 18 shows:

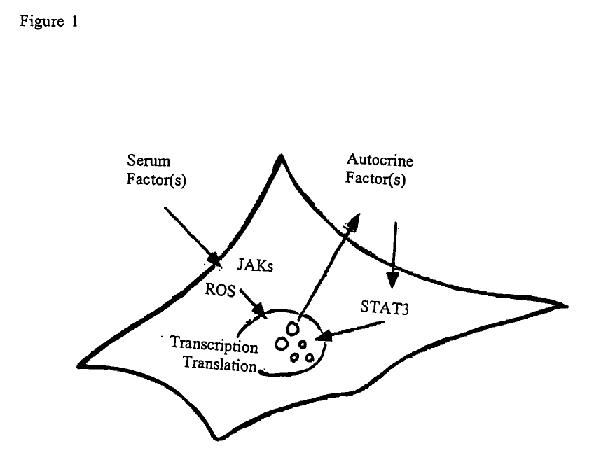

OdDHL blocks the autocrine release of mitogens from BC cells (a) Equal numbers of BR293 cells were cultured in MEM alone, serum-free MEM conditioned by serum-stimulated BR293 cells for 2 hours, or the same supplemented with 5% FCS, as indicated. After 24 hours [³H]-thymidine was added to the medium. After a further 18 hours, cells were harvested and analysed for incorporation of ³H. Error bars denote SD (n=4). (b) BR293 cells were serum-starved (lane 1) or starved and stimulated with 10% FCS for 2 hours directly (lane 2) or after pretreatment with α-amanitin (1004/ml) for 2.5 hours (lane 3) or actinomycin D (10 g/ml) for 10 min (lane 4). Nuclear extracts were prepared and analysed for STAT1 and STAT3 complexes as described in the legend to FIG. 1b. (c) BR293 cells were serum-starved (lane 1) or starved and stimulated with 10% FCS alone for 2 hours (lane 2), with serum-free CM alone for 15 min (lane 5) or in the presence of increasing 100 μM AG490 (lanes 3 and 6) or 100 μM OdDHL (lanes 4 and 7). Cell lysates were prepared and analysed by Western blotting as described in the legend to FIG. 1a. (d) BR293 cells were stimulated with 10% FCS in the presence of 100 μM OHHL (lane 1) or OdDHL (lane 2). After 2 hours the cells were washed and incubated for a further 2 hours in serum-free MEM. The conditioned medium (CM) was then used to stimulate fresh serum-starved cells for 15 min, as indicated. Lysates were prepared from all the cells and analysed by Western blotting as described in the legend to FIG. 1a.

FIG. 19 shows:

ERKs are unaffected by OdDHL (a) BR293 cells were serum-starved (lane 1) or starved and stimulated with TPA for 30 min alone (lane 2) or in the presence of increasing concentrations of OdDHL (lanes 3-6) or 100 μM OHHL (lane 7). Cell lysates were prepared and analysed by Western blotting for ERK phosphorylation with an antibody specific for phospho-ERK½ (upper panel) and for ERK content with an antibody against ERKs (lower panel). (b) BR293 cells were serum-starved (lane 1) or starved and stimulated with Anisomycin for 30 min alone (lane 2) or in the presence of increasing concentrations of OdDHL (lanes 3-6) or 100 μM OHHL (lane 7). Cell lysates were prepared and analysed by Western blotting for JNK/SAPK phosphorylation with an antibody specific for phospho-JNKs (upper panel) and for SAPK/JNK content with an antibody against JNKs (lower panel). (c) BR293 cells were treated as described in (b) and lysates were analysed by Western blotting for p38^{MAPK} phosphorylation with an antibody specific for phospho-p38 (upper panel) and for p38 content with an antibody against p38 (lower panel).

FIG. 20 shows:

OdDHL potentiates STAT3 activation by EGF (a) MDA-MB-468 cells were serum-starved (lanes 1-3) or starved and stimulated with EGF for 15 min (lanes 3-6) alone or in the presence of OdDHL at 10 μM (lanes 2 and 5) or 100 μM (lanes 3 and 6). EGF-R was immunoprecipitated from cell lysates and analysed by Western blotting tyrosine phosphorylation with an anti phosphotyrosine antibody (upper panel, top) and EGF-R content with an anti-EGF-R antibody (lower panel, top). Cell lysates were analysed in parallel for STAT3 phosphorylation with an antibody specific for STAT3 phosphorylated on Y705 (upper panel, bottom) and for STAT3 content with an antibody for STAT3 (lower panel, bottom). (b) Nuclear extracts were prepared from serum-starved MDA-MB-468 cells (lanes 1 and 2) or starved cells treated with EGF alone (lanes 3 and 4) or EGF and 100 μM OHHL (lanes 5 and 6) or EGF and 100 μM OdDHL (lanes 7 and 8), and analysed for STAT1 and STAT3 DNA-binding activity by EMSA with a radio-labelled probe corresponding to the M67 SIE. The inclusion of an anti-STAT3 antibody in duplicate binding reactions (even lanes) identifies STAT3-containing heterodimers (3:1), as indicated to the right of the panel. (c) COS-1 cells were transfected with an expression vector for STAT3, a STAT3-responsive luciferase reporter (SIE2-luc) and a control gene for β-galactosidase. After recovery and culture in starvation medium for 18 hours, cells were stimulated with EGF alone or together with AHLs or specific tyrosine kinase inhibtors, as indicated. After 6 hours cells were harvsted and reporter gene expression analysed. Results are normalised against β-galactosidase values and expressed as averages +/−SD (n=3).

FIG. 21 shows: In the top section: BR293, MCF-7 and MDA-MB-468 cells were serum-starved (lanes 1, 6 and 11) or starved and stimulated with 10% FCS for 2 hours, either alone (1 lanes 2, 7 and 12) or in the presence of 200 nM Wortmannin (lanes 3, 8, 13) 100 μM OdDHL (lanes 4, 9, 14) or OHHL (lanes 5, 10, 15). Cell lysates were then prepared and analysed by Western blotting for Akt/PKB phosphorylation with an antibody specific for Akt/PKB phosphorylated on S473 (p-Akt, upper panel) and subsequently for total Akt/PKB content with an antibody for Akt/PKB (lower panel).

In the bottom section of FIG. 21, BR293 cells were serum-starved (lane 1) or starved and stimulated with 10% FCS for 2 hours alone (lane 2) or in the presence of increasing concentrations of OdDHL (lanes 3-6) or 100 μM OHHL (lane 7) or 200 nM Wortmannin (lane 8). Cell lysates were prepared and analysed by Western blotting for Akt/PKB phosphorylation with an antibody specific for Akt/PKB phosphorylated on S473 (upper panel) and subsequently for total Alt/PKB content with an antibody for Akt/PKB (lower panel).

In this first section of disclosure and exemplification, the materials and methods used were as described in the following section entitled “MATERIALS AND METHODS—1”.

A number of abbreviations are used in this disclosure that are well-known and obvious to those skilled in the art. The following abbreviations are also well-known, but for clarity are defined here:

| | |
|---|---|
| BC | Breast Carcinoma |
| JAK | Janus kinase |
| OdDHL | N-(3-oxo-dodecanoyl)-L-homoserine lactone |
| OHHL | N-(3-oxohexanoyl)-L-homoserine lactone |
| AHL | N-acyl-L-homoserine lactone |
| EGF | Epidermal Growth Factor |

ErbB and STAT Protein Expression in BC Cell Lines

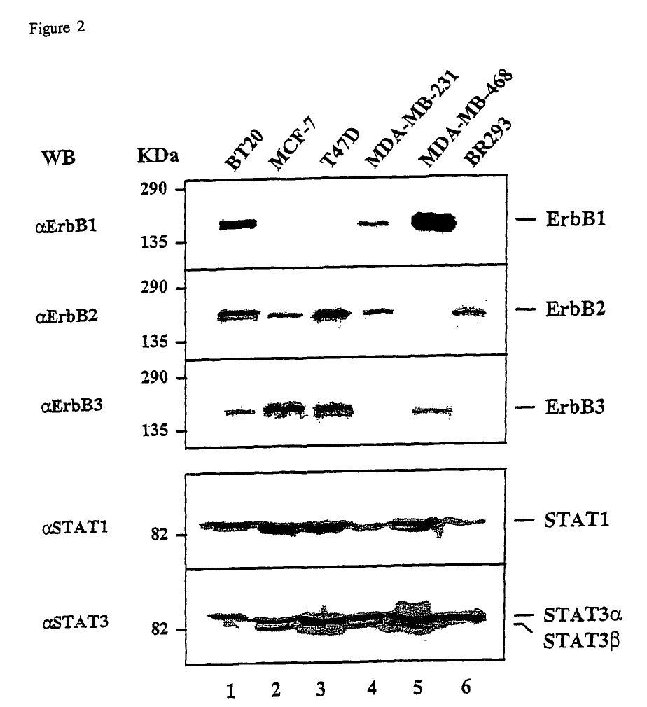

Initially, the expression levels of ErbB proteins in six BC-derived cell lines were compared by immunoblotting. As shown in FIG. 2 (upper panel), ErbB1 was strongly expressed in MDA-MB-468 cells, moderately expressed in BT20 cells, weakly expressed in MDA-MB-231 cells and undetectable in the other three cell

lines (MCF-7, T47D and BR293). However, MCF-7 and T47D cells have been shown previously to express low levels of surface ErbB1, indicating that the limit of detection must lie above 10,000 receptors per cell. ErbB2 was expressed at a similar level in all of the cell lines, with the exception of MDA-MB-468, in which it was undetectable. Expression of ErbB3 was also analysed and found to

be moderate in MCF-7 and T47D, weak in BT20 and MDA-MB468 and absent from MDA-MD-231 and BR293 cells. In contrast, the expression of STAT1 and STAT3 proteins in these cells showed much less variation (FIG. 2, lower panel). BR293 cells alone express low levels of STAT1 proteins (lane 6). Both isoforms of STAT3 (STAT3a and B) are expressed in all the cell lines but the 13 isoform is expressed at a lower level in BT20 and BR293 cells (lanes 1 and 6). Thus, these six BC-derived cell lines exhibit five different profiles of ErbB expression, whereby only those exhibited by MCF-7 and T47D cells are similar. However, they express comparable levels of STAT1 and STAT3 proteins.

Tyrosine Phosphorylation of ErbB Proteins in BC Cell Lines

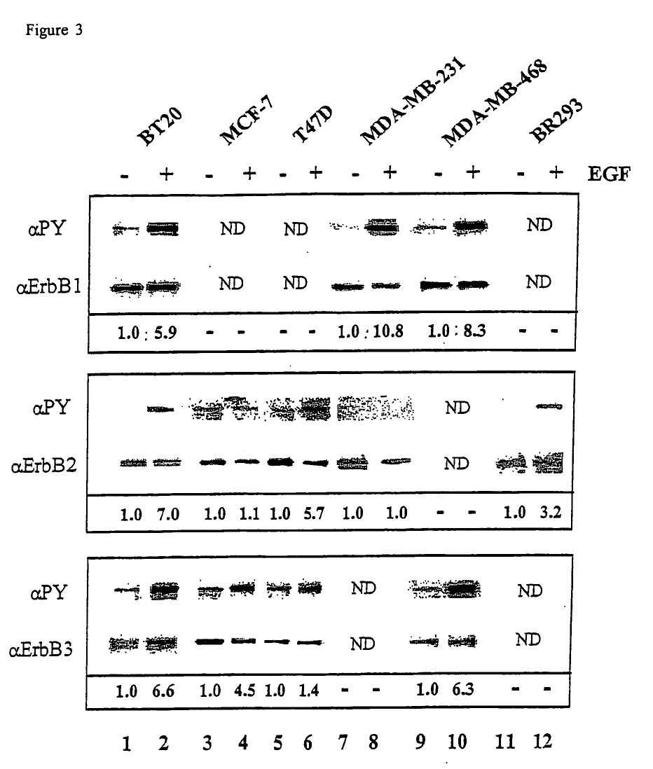

The activity of ErbB proteins is a consequence of their tyrosine phosphorylation status. Accordingly, tyrosine phosporylation of ErbB proteins was analysed, in those cells in which they could be detected (FIG. 3), by immunoprecipitation and subsequent detection with a phosphotyrosine-specific antibody (PY20). InBT20, MDA-MB-231 and MDA-MB-468 cells, tyrosine phosphorylation of ErbB 1 is weak or undetectable in normally growing cells (FIG. 3, upper panel), but, as expected, it is induced (5.9, 10.8 and 8.3 fold, respectively) upon treatment of cells with EGF.

Tyrosine phosphorylation of ErbB2 is detectable in normally growing MCF-7 and T47D cells but not in the other cell lines. In MDA-MB-231 cells, EGF treatment does not elicit an increase in ErbB2 tyrosine phosphorylation, even though ErbB1 is expressed (see FIG. 3) and becomes phosphorylated itself. However, in T47D and BR293 cells, which both lack ErbB1 (see FIG. 3), stimulation of ErbB2 tyrosine phosphorylation by EGF is apparent (5.7 and 3.2 fold respectively).

ErbB3 tyrosine phosphorylation is also observed under normal growth conditions in all four cell lines in which it is expressed. Moreover, in those cell lines in which ErbB1 is co-expressed, tyrosine phosphorylation of ErbB3 is induced by EGF (6.6 and 6.3 fold). In summary, although the variations in ErbB protein expression among the cell lines precludes direct quantitative comparison, those cells expressing ErbB1 display low levels of tyrosine phosphorylation on ErbB2 and ErbB3 proteins that become elevated following stimulation by EGF. Conversely, cells lines that lack ErbB 1 show constitutive levels of tyrosine phosphorylation on ErbB2 and ErbB3 that remain unchanged or increase only marginally when cells are treated with EGF.

STAT Activation in BC Cell Lines

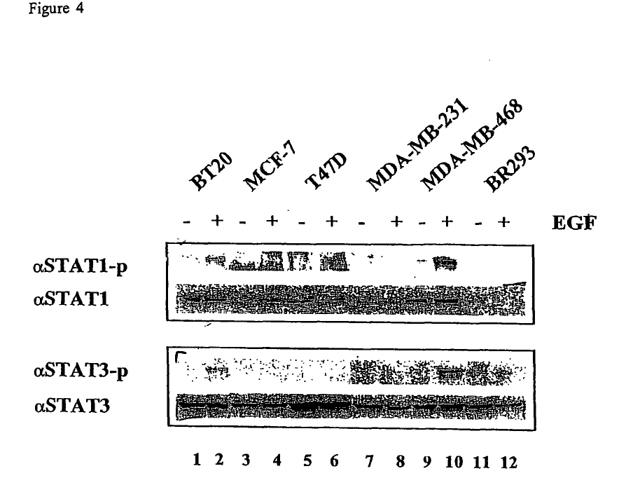

The phosphorylation of STAT1 and STAT3 proteins was also examined in all six cell lines with phospho-specific antibodies for each protein. As shown in FIG. 4 (upper panel), tyrosine phosphorylation of STAT1 was undetectable in serum-starved cells, but was stimulated in BT20 and MDA-MB468 cells following EGF treatment (lanes 2 and 10). As already seen in FIG. 2, BR293 cells express low levels of STAT1. A low level of STAT3 tyrosine phosphorylation could be seen in serum-starved BR293 cells (lower panel, lane 11), while in STAT3 immunoprecipitates probed with an anti-phosphotyrosine antibody phosphorylated STAT3 was detected in all six cell lines (result not shown). Following EGF stimulation, however, tyrosine phosphorylation of STAT3 also increased in BT20 and MDA-MB-468 cells (lanes 2 and 10), mirroring the behaviour of STAT1. Because EGF-induced tyrosine phosphorylation of ErbB1 also occurs in MDA-MB-231 cells (see FIG. 3), the failure to induce STAT3 tyrosine phosphorylation is likely to be a consequence of the lower level of ErbB1 expression in this cell line (see FIG. 2).

The function of STAT proteins depends on their DNA-binding ability, for which tyrosine phosporylation and dimerisation are prerequisites. Initially, whole cell extracts prepared from BC cells were analysed for STAT binding activity with a cognate binding element derived from the c-fos SE. In extracts of serum-starved MDA-MB-468 cells, in which ErbB1 is highly expressed, a low level of heterogeneous DNA binding was detected (FIG. 5, lane 1), which could be attributed, by supershift assay with anti-STAT antibodies, to STAT3 (lane 3). Control experiments confirmed that the anti-STAT3 antibody does not generate the supershifted complex (3SS), seen here and in subsequent figures, in the absence of DNA-binding by STAT3 (data now shown). After stimulation of the cells with EGF, DNA-binding was much enhanced and several additional complexes were detected (lane 4) that contained STAT1 and STAT3, as evidenced by supershift assay with specific antibodies (lanes 5 and 6).

In parallel experiments with BT20 cells, which also express ErbB1, EGF induced the formation of a similar set of complexes (FIG. 5, lanes 10-12). However, under the same experimental conditions, STAT complex formation was weaker, which may reflect the lower ErbB 1 expression in these cells (FIG. 2). Furthermore, we did not detect the induction of STAT complexes by EGF in MDA-MB-231 cells, which express even less ErbB 1 (result not shown). When this experiment was carried out with cells lacking ErbB1 (BR293), weak DNA binding by STAT3 was again detected in extracts of serum-starved cells, but EGF failed to stimulate the formation of additional STAT-DNA complexes (FIG. 5, lanes 16-18). Thus, acute stimulation of STAT1 and STAT3 DNA-binding activity in response to EGF correlates directly with ErbB 1 expression in BC cells.

Acute STAT Activation Requires ErbB1 and JAK Kinase Activity

To confirm that the acute activation of STAT DNA-binding in response to EGF was dependent upon ErbB1 kinase activity. EGF stimulation was repeated in the presence of the quinazoline inhibitor PD 153035. Pre-treatment of MDA-MB-468 cells with 100 nM PD 153035 for 30 minutes inhibited tyrosine phosphorylation of ErbB1 (FIG. 6a) and abrogated the induction of SIE-bound STAT complexes by EGF (FIG. 6b). However, PD 153035 had no effect on the weak, heterogeneous DNA-binding by STAT3 detected by supershift assay in extracts from serum-starved cells (lane 3 and lanes 9, 12, 15, 18, 21). Thus the acute activation of STAT DNA-binding by EGF requires ErbB 1 kinase activity.

The acute induction of STAT DNA-binding activity was examined in cells treated with the JAK inhibitor AG490 (34). As shown in FIG. 6c, 100 μM AG490 abolished STAT activation by EGF in BT20 and MDA-MB-468 cells. The weak, STAT3 DNA-binding was again unaffected (compare lane 2 with lane 6 and lane 7 with lane 9). Thus, acute stimulation of STAT DNA-binding requires both ErbB1 and JAK kinase activity, whereas the weak STAT3 DNA-binding requires the activity of neither.

A basal level of STAT3 DNA-binding similar to that in BT20 cells was also seen in serum-starved MDA-MB-231 and BR293 cells (FIG. 6d) and could be detected in MCF-7 and T47D cells (not shown). In all cases, the complexes persisted in cells from which serum had been withdrawn for up to three days (FIG. 6d, lane 6 and results not shown).

Serum Induces Elevated STAT3 Activity via an Autocrine Signal

When serum-starved BR293 cells, which lack ErbB1, were returned to full medium, an increase in STAT3 tyrosine phosphorylation over a 2 hour time course was observed (FIG. 7a, upper panels). Tyrosine phosphorylation of JAK2 was also stimulated by serum over the same period (lower panels) and both effects were blocked by AG490. As shown in FIG. 7b, STAT DNA-binding activity in nuclear (left panel) and whole cell extracts (right panel) also increased, reaching a peak at 2 hours. Importantly, the STAT complexes observed in whole cell extracts were also present in nuclear extracts, with the exception of the STAT3 complexes detected in unstimulated cells.

Compared to the rapid, acute induction by EGF, the kinetics of STAT activation in response to serum was delayed, indicating that the upregulation of STAT DNA-binding by serum involves the autocrine/paracrine mechanism now claimed.

Serum-starved BR293 cells were stimulated with 10% foetal calf serum (FCS) and, after 2 hours, half the cells were harvested while the other cells were washed thoroughly and incubated for a further 4 hours in serum-free medium. This medium was then transferred to fresh, serum-starved BR293 cells, which were incubated for a further 15 minutes. Nuclear extracts were made from all the cells and analysed for STAT DNA-binding. As shown in FIG. 7c, STAT1 and STAT3 DNA-binding was stimulated after 2 hours by 10% FCS (lanes 3 and 4). In contrast, serum-free conditioned medium from cells incubated previously with 10% FCS for 2 hours stimulated STAT3 DNA-binding after 15 minutes (lanes 5 and 6). Similarly, conditioned medium from MDA-MD-468 cells cultured for 2 hours with 10% FCS was able to stimulate STAT3 DNA-binding in BR293 cells within 15 minutes (result not shown). Treatment of BR293 cells with conditioned medium also induced tyrosine phosphorylation of STAT3 within 15 minutes, whereas EGF treatment did not (FIG. 7d). Demonstrating that BC cells cultured in 10% FCS release factors that stimulate tyrosine phosphorylation of STAT3 and its consequent DNA-binding activity.

As BR293 cells do not express ErbB1, the involvement of ErbB1 in the serum-dependent activation of STAT3 is unlikely. Consistent with this inference, when FCS was applied to serum-starved MDA-MBA468 cells pre-treated with PD 153035, the delayed serum stimulation of STAT3 DNA-binding was not affected (result not shown). The role of JAKs in the serum-depedent activation of STAT3 was further assessed by pre-treating BR293 cells with 100 μM AG490 for 30 minutes which completely blocked STAT3 activation (FIG. 8a, lanes 5 and 6). However, when conditioned medium from BR293 cells was applied to serum-starved cells treated with AG490, no inhibition was observed (lanes 17 and 18). Showing that primary signal mediating STAT3 activation by serum requires JAK activity, whereas the secondary autocrine signal acts independently of JAKs. MDA-MB468 cells growing in full medium were treated with AG490 for 24 hours, as shown in FIG. 8b, this reduced the elevated, serum-dependent level of STAT3 activity to a constitutive basal level.

Taken together, the preceding results distinguish three distinct levels of STAT activity in BC cells, as manifested by DNA-binding. Firstly, in cells expressing ErbB1, several STAT-DNA complexes can be induced acutely by EGF, which is dependent upon the kinase activity of both ErbB1 and JAKs. Secondly, anintermediate level of DNA-binding is induced by serum via an autocrine mechanism involving JAKs but not ErbB1. Thirdly, a weak, constitute level of DNA-binding by STAT3, which is independent of ErbB1 and JAKs, is detected in whole cell extracts from serum-starved cells and persists for up to 3 days.

Inhibition of BC Cell Growth

MDA-MB-468 and MCF-7 cells were transfected with expression vectors for STAT3 and two trans-dominant negative mutants thereof. In each instance, the dominant negative mutants caused a 25-30% decrease in the growth of transfected cells over 4 days (FIG. 9, top left panel). As only a proportion of the cells was transfected, this result probably under-estimates the effect of dominant-inhibitory STAT3 mutants on BC cell growth. STAT3 is therefore crucial in the proliferation of these BC cell lines. The effects of JAK inhibition on BC cell growth was measured. As shown in FIG. 9 (top right panel), treatment of MDA-MB468 cells and BR293 cells with AG490 for 24 or 48 hours had a dramatic effect on cell growth, reducing cell proliferation by >75% over 48 hours. Thus, JAK function is important for BC cell proliferation. As JAKs are involved in both the acute and intermediate levels of STAT3 activity, the effect on cell growth of two specific ErbB1 inhibitors, PD 153035 and PD 168393 was also measured. Treatment of MDA-MB-468 and BR293 cells with either reagent had no effect on their proliferation over 48 hours (lower panels). To demonstrate that ErbB1 was indeed inhibited, MDA-MB-468 cells cultured and treated with PD 168393 in parallel were stimulated at different time points with EGF for 15 minutes and tyrosine phosphorylation of ErbB1 was measured. PD 168393 completely inhibited ErbB1 tyrosine kinase activity over 24 hours (FIG. 9, inset). This data shows that BC cell proliferation correlates with STAT3 activity which is maintained by the serum-dependent autocrine/paracrine pathway now claimed.

OdDHL Blocks Serum Stimulation of STAT1 and 3.

Serum-starved BR293 cells were pre-treated with 200 μM OdDHL (active) or OHHL (inactive) for 30 min. and then stimulated with serum for 2 h. Cells were lysed and the DNA-binding activity of STAT1 and 3 was examined by EMSA. As shown in FIG. 10 (left panel), pre-incubation of the cells with OdDHL, but not OHHL, prevented serum induction of STAT1 and 3 complexes. Treatment of serum-starved BR293 cells with either AHL alone for up to 2 h did not induce STAT complex formation (right hand panels). Similarly, as shown in FIG. 11 (upper panel), pre-incubation of BR293 cells with OdDHL, but not OHHL or an unrelated signal molecule (PQS), blocked the serum-induced phosphorylation of STAT3. (This panel is the first part of an experiment also shown in FIG. 13.)

None of the three compounds had any noticeable effect on the activation of the ERK Mitogen-Activated Protein Kinase (MAPK) cascade by TPA, as indicated by their failure to prevent phosphorylation of ERK½ (middle panel). This indicates that the action of OdDHL on STAT activation is not part of an unspecific, pleiotropic effect. Other small molecules also inhibited serum induction of STAT1 and STAT3 DNA-binding activity, for example scavengers of reactive oxygen species (ROS) such as N-acetyl cysteine (NAC) and diphenylene iodonium chloride (DPI) and the JAK inhibitor AG490 (see Figure 11, lower panel).

OdDHL Blocks Autocrine Factor Release from BC Cells

A titration of OdDHL revealed that its IC50 for the inhibition of serum-induced STAT1 and STAT3 DNA-binding activity lies between 50-100 μM (FIG. 12, left hand panel). OdDHL, but not OHHL, also blocks the activation of STAT1 and STAT3 DNA binding by H₂O₂, which increases intracellular ROS, but not the activation of STAT 3 DNA binding by conditioned medium (CM) from BR293 cells (right hand panels). This indicates that OdDHL inhibits the STAT3 activation pathway upstream of autocrine factor release (see FIG. 1). Autocrine factor release is frequently associated with de novo gene expresion and protein synthesis. As shown in FIG. 13 (upper panel), both α-amanitin and Actinomycin D (inhibitors of transcription) reduce the levels of STAT1 and STAT3 activation by serum whereas methanol (the vehicle) has no inhibitory effect, indicating that this pathway involves gene expression.

If OdDHL can block autocrine factor release, then conditioned medium from treated cells should not elicit STAT3 activation. Indeed, CM from serum-stimulated BR293 cells pre-treated with OdDHL was unable to stimulate STAT3 phosphorylation, whereas CM from serum-stimulated cells pre-treated with OHHL or PQS could do so (FIG. 13, lower panels).

OdDHL blocks BC cell oroliferation and induces apoptosis. BR293 cells were grown in full medium (10% FCS) in the absence or presence of OdDHL or the control OHHL. After 24 h, cell were harvested, stained with DAPI and examined by fluorescence microscopy. OdDHL induced DNA condensation and nuclear fragmentation whereas untreated or OHHL-treated cells remained viable (FIG. 14, left hand panels). The effect of OdDHL on proliferation was also apparent: cells cultured in the presence of OdDHL for 48 h grew poorly or not at all, but control cells plated at the same density reached confluence (right hand panels).

Taken together, these results show that serum-dependent STAT3 activity is required for BC cell proliferation and that biologically active AHLs such as OdDHL block activation of STAT1 and 3, thereby inhibiting BC cell proliferation.

In the following section of description and exemplification, the materials and methods referred to are described fully in the section entitled “Materials and Methods—2” that follows.

The human pathogen Pseudomonas aeruginosa uses quorum-sensing signal molecules (QSSMs) to regulate virulence gene expression. It has been shown that such molecules are also able to suppress host immune responses of the type commonly associated with auto-immune disease, although the mechanism of action is obscure. However, regulation of immune function is known to involve STAT proteins.

Here we have explored the possibility that QSSMs of P. aeruginosa are able to modulate STAT activity in the context of BC cell growth. We show that constitutive STAT3 activity in proliferating human BC cells is down-regulated by the QSSM N-(3-oxododecanoyl)-L-homoserine lactone OdDHL, resulting in apoptotic cell death. These results support the notion of OdDHL as a bioactive molecule in eukaryotic systems and as a paradigm for a novel class of antiproliferative molecules.

T cell responses to immune challenge are orchestrated by a complex array of cytokines with diverse and often selective effects on their target cells, controlling, among other things, cell survival and proliferation. Inextricably linked to cytokine action are intracellular signalling pathways that involve Signal Transducer and Activator of Transcription (STAT) proteins and a family of protein tyrosine kinases referred to as Janus Kinases (JAKs) (20). Activated cytokine receptors provide scaffolds upon which STATs are phosphorylated by JAKs, whereupon STATs translocate to the nucleus and up-regulate the expression of target genes, which include genes for numerous cytokines (10, 17).

The intimate associations between pathogen and host make it highly likely that the former have evolved subtle and selective strategies to subvert the immune systems of the latter.

Notably, the widely used immune-suppressant drugs Cyclosporin A and Rapamycin are both naturally occurring compounds. In this context it has been shown recently that quorum-sensing signal molecules (QSSMs) from Pseudomuonas aeruginosa are able to suppress immune responses of the type commonly associated with auto-immune disease (25). In contrast, more recent data suggests that these molecules may have pro-inflammatory activity (23). However, the mechanism underlying these responses is obscure and the molecular target for OdDHL remains to be identified. Given their involvement in cytokine-mediated events, elements of the JAK/STAT pathway would appear to offer prime targets for pathogens aiming to evade or inactivate the immune systems of their hosts.

STAT proteins are implicated in cellular processes distinct from those regulating the immune system. For example, STAT3 plays a role in driving cell proliferation and counteracting differentiation signals (2), while a STAT3 mutant that dimerises in the absence of tyrosine phosphorylation is constitutively active and functions as an oncogene (4). Moreover, the proliferation of a range of tumour-derived cells, notably of Breast Carcinoma (BC) origin, has been shown by several groups to depend on the constitutive activity of STAT3 (5, 7, 11).

Given the importance of the JAK/STAT pathway for immune function and of STAT3 for cell proliferation, we decided to explore the possibility that AHLs might modulate STAT signalling in the context of cell proliferation. Here we show that STAT1 and STAT3 activities in proliferating human BC cells are down-regulated by N-(3-oxododecanoyl)-L-homoserine lactone (OdDHL), the major QSSM of P. aeruginosa (27) (16), resulting in apoptotic cell death. However, in cells stimulated by EGF the acute activation of STAT3 is augmented by OdDHL. Our findings indicate that OdDHL is a bioactive molecule in eukaryotic systems and a paradigm for a novel class of antiproliferative molecules. They also raise the possibility that in order to serve disparate roles STAT3 may be partitioned into two functional populations, whereby disruption of one automatically augments the other.

Inhibition of STAT Activity by Bacterial QSSMs

As STAT3 activity appears to be important for BC cell proliferation, it was of interest to identify inhibitors of STAT function and test their consequent effects on BC cell growth. Among the potential inhibitors chosen for analysis were bacterial QSSMs, some of which have recently been shown to influence aspects of host immune function, in which the JAK/STAT signalling pathway is notionally involved (25). Pretreatment of BR293 cells for 15 minutes with increasing concentrations of OdDHL inhibited serum-induced STAT3 tyrosine phosphorylation at 100 μM (FIG. 15a, lane 6). In contrast, the short chain analogue N-butanoyl homoserine lactone (OHHL), which lacks immune-modulatory activity, had only a slight effect at the same concentration (lane 7). Similar results were obtained with MDA-MB-468 cells (FIG. 15a, lower panel).

The effects of AHLs on STAT3 DNA binding were also monitored in parallel. Nuclear extracts were prepared and assayed with a high affinity STAT1/STAT3 binding site (M67 SIE 9 (29)) and STAT3-containing complexes were identified by including a STAT3-specific antibody in duplicate binding reactions. In this assay OdDHL was seen to inhibit DNA binding by STAT3 at 50 μM (FIG. 16b, lanes 9 and 10), while again OHHL had only a marginal effect at 1001M (lanes 13 and 14). The lower IC₅₀ for DNA-binding suggests that the negative impact of OdDHL on STAT phosphorylation may be an indirect consequence of OdDHL acting primarily on a subsequent event in the STAT activation mechanism. Again, similar results were obtained with MDA-MB-468 cells, except that only the STAT3 homodimer was detected (FIG. 15b, lower panel).

It is noteworthy that the small increase in STAT3 phosphorylation at 50 μM OdDHL is observed consistently in BR293 cells, as is the increase in DNA binding by STAT1 and STAT3 at 20 μM. In MDA-MB-468 cells, an increase is also observed and is apparent at 10 μM OdDHL. This may be explained by OdDHL having opposite effects on two signal pathways converging on STATs (see also below). In summary, OdDHL, but not OHHL, severely impairs STAT3 activation in serum-stimulated cells.

OdDHL Inhibits BC Cell Proliferation

Treatment of BC cells with OdDHL had a marked effect on their proliferation. As shown in FIG. 16a, BR293 cells treated with vehicle (DMSO) or OHHL reached confluence in 48 hours, whereas those exposed to OdDHL grew poorly or not at all. Indeed, OdDHL markedly inhibited the proliferation of three tumorigenic BC cell lines (MCF-7, BR293 and MDA-MB-468) by 70%-80% over a 48-hour period. However, its effect on the proliferation of non-tumorigenic breast epithelial cells (MCF-10F), which were previously shown to be insensitive to the JAK inhibitor AG490 (5, 11), was slight (FIG. 16b). We also examined the effects of OdDHL and OHHL on transformed human and simian cell lines. Growth of HEK293 cells, which have constitutive STAT3 activity, was sensitive to treatment with OdDHL, whereas proliferation of COS1 cells, which are known to express low levels of STAT proteins (22), was unaffected (FIG. 16c). These findings again link constitutive STAT3 activity to cell proliferation.

OdDHL Induces Apoptosis in Proliferating BC Cells

The low numbers of BC cells surviving for 48 hours in the presence of OdDHL suggested that they might be undergoing apoptotic cell death. To assess this possibility, cell integrity was analysed In growing MCF-7 and MDA-MB-468 cells treated with OdDHL for 24 hours, staining with 4′,6′-diamidino-2-phenylindole (DAPI) revealed evidence of nuclear disruption, possibly indicative of apoptosis, whereas control cells or cells treated similarly with OHHL were unaffected (FIG. 17a). Therefore the integrity of poly ADP-ribose polymerase (PARP), a well-characterised target for caspases, was also examined. Although BR293 cells express low levels of PARP, the characteristic cleavage fragment was detected in cells treated with OdDHL (FIG. 17b, lane 2). OdDHL also induced PARP cleavage in MCF-7 cells (lane 4) and in MDA-MB-468 cells to the same extent as etoposide (compare lanes 6 and 7). Taken together, these results indicate that STAT3 inhibition by OdDHL causes apoptosis in proliferating BC cells.

OdDHL Downregulates Autocrine Release from BC Cells

Serum has been shown to stimulate the release of an autocrine factor(s) that contributes to STAT3 activation in BC cells (11). Autocrine secretion of Prolactin has previously been reported to activate JAK2 and hence ErbB2 in BC cells (28). In addition, angiotensin II was found to stimulate autocrine release of IL-6 from rat cardiomyocytes, resulting in elevated STAT activity (19). However, as discussed previously (11), several criteria appear to distinguish these mechanisms from the autocrine-mediated STAT3 activation pathway in BC cells.

As shown in FIG. 18a, BR293 cells cultured in low serum undergo DNA synthesis, as measured by ³H-thymidine incorporation. However, when CM was supplemented with 5% serum, the level of DNA synthesis doubled, indicating that CM contains one or more mitogens released by BR293 cells.

Given the time delay between serum stimulation and the resultant activation of STAT3 (11), it was conceivable that the autocrine process involved de novo gene expression. Pretreatment of cells with α-amanitin for 2.5 hours or with Actinonycin D for 10 minutes blocked the stimulation of DNA binding by STAT1 and STAT3 (FIG. 18b, lanes 3 and 4), which is consistent with this notion.

The JAK inhibitor AG490 inhibits BC cell proliferation (7) and interferes with STAT3 activation by serum but not CM (11). Similarly, pretreatment of cells with OdDHL prevented the delayed STAT3 phosphorylation in response to serum (FIGS. 18c, lane 4; see also FIG. 15a) but not the rapid response to CM (FIG. 18c, lane 7). However, as shown in FIG. 18d, CM from serum-stimulated cells that had been pretreated with OdDHL failed to induce STAT3 phosphorylation (lane 4) while CM from cells pretreated with OHHL was active (lane 3), indicating that OdDHL-treated cells fail to release the active autorcrine factor.

Influence of OdDHL on MAPK Cascades

The effects of OdDHL on STAT3 activation by serum prompted us to monitor its effects on other signalling pathways. One downstream consequence of serum stimulation of cells is the activation of MAPK cascades, reflected by the phosphorylation of Extracellular signal-regulated Kinases (ERKs) and, in some cases, cJun N-terminal Kinases/Stress-Activated Protein Kinases (JNK/SAPK) and p38-family MAPKs (9, 18). We therefore measured the effects of OdDHL on MAPK cascades activated by pathway-specific stimuli.

Neither OdDHL nor OHHL had any effect on the activation of ERKs in response to TPA (FIG. 19a) or serum (result not shown). In contrast, OdDHL, but not OHHL, caused a modest but reproducible inhibition of p46/p54 JNK/SAPK (FIG. 19b) and p38^{MAPK} phosphorylation (FIG. 19c) in cells treated with anisomycin, a well-established stress agonist. However, it is unlikely that this inhibition of stress-activated MAPKs contributes to the anti-proliferative effects of OdDHL

OdDHL Enhances STAT Activation in Response to EGF

OdDHL blocks STAT3 activity in proliferating BC cells and precipitates cell death by apoptosis, providing further evidence for a link between STAT3 and cell proliferation.

However, it remained to be seen if OdDHL also blocks STAT3 activation in response to acute stimulation. Although BR293 cells lack the EGF receptor (EGF-R) and do not respond to EGF, MDA-MB-468 cells, which are equally susceptible to OdDHL (FIG. 15), express high levels of the receptor (11). We therefore tested the influence of OdDHL on the activation of STAT3 by EGF in MDA-MB-468 cells, which express the EGF-R. As shown in FIG. 20, the outcome was markedly different. OdDHL alone at 100 μM caused an increase in detectable tyrosine phosphorylation of the EGF-R in unstimulated cells (FIG. 20a, lane 3) and potentiated receptor tyrosine phosphorylation in response to EGF stimulation several fold, even at lower concentrations (101M) (compare lanes 5 and 6 with lane 4).

Similarly, STAT3 phosphorylation in response to EGF was augmented by co-treatment of cells with 100 μM OdDHL (FIG. 20a, lower panels), while in DNA-binding assays activation of STAT3 was seen to be enhanced by OdDHL but unaffected by OHHL (FIG. 20b, compare lanes 7 and 8 with lanes 3-6). Moreover, in COS-1 cells transfected with an expression vector for murine STAT3, the 5-6 fold stimulation of STAT3-dependent reporter gene expression by EGF was augmented by co-treatment of cells with OdDHL (FIG. 20c).

In contrast, OHHL had no effect (not shown). The involvement of the EGF-R and JAKs in these events was confirmed with the inhibitors PD153035 and AG490 respectively. Thus, in stark contrast to its effects on serum-dependent STAT3 activity, OdDHL has a positive effect on STAT3 activation in response to EGF.

Discussion

STAT proteins are fundamentally involved in implementing the changes in gene expression that coordinate numerous biological programmes, such as haematopoiesis, embryogenesis and immune responses. STAT3 in particular has also been linked to cell proliferation and survival and shown to possess oncogenic potential (2, 3). Based on the premise that pathogens gain advantage by subverting host immune systems, in which STATs play pivotal roles, we explored the possibility that QSSMs of P. aeruginosa can modulate STAT activity. We found that OdDHL, but not OHHL, is able to potentiate acute stimulation of STAT3 by EGF, but to down-regulate STAT3 activity and induce apoptosis in the context of proliferating BC cells.

Role of STAT3 in BC Cell Proliferation

A large body of evidence implicating STAT3 as a positive regulator of proliferation in a range of tumour tissues has accumulated. Evidence for the role of STAT3 included the effects of dominant-inhibitory STAT3 mutants, which were found to reduce proliferation of several tumour cell types, counteract transformation by several oncogenes and exert a negative-selection on the establishment of stable cell lines in which they were expressed (reviews in (26).

STAT3 is involved in the expression of several proteins that participate in cell cycle control. It appears to mediate the induction of c-myc in response to growth factors including IL-6 and various oncogenes including v-src and v-abl (reviewed in (8), which would contribute to G₁-S progression. In addition, Pim1 and Pim2 have been identified as STAT3-responsive genes. Pim1 encodes a serine/threonine kinase that phosphorylates and activates Cdc25A, a major regulator of cyclin-dependent kinases. In the absence of STAT3 activity constitutive expression of both Pim1 and c-Myc was shown to be required for cell cycle progression (14, 21).

STAT3 can also influence the balance between survival and apoptotic signals. Several lines of evidence indicate that pro-survival Bcl-family members are upregulated by STAT3. For example the high levels of Bcl-xL expressed in the Fas-resistant myeloma cell line U266 are reduced upon STAT3 inactivation, whereupon the cells undergo apoptosis (6). These findings highlight the role of STAT3 as a positive regulator of the cell cycle and anti-apoptotic signalling in at least a subset of human cell types.

Distinct Pathways for STAT3 Activation

The most intriguing of our findings relates to the converse effects of OdDHL on two distinct modes of STAT3 activation. Whereas the serum-dependent STAT3 activity was abrogated by OdDHL, acute activation of STAT3 in response to EGF was enhanced, an effect apparent at the level of EGF-R phosphorylation. STAT3 activation in response to EGF is known to require the kinase activities of its receptor and JAK2, whereas serum stimulation involves only the latter (11). OdDHL is therefore unlikely to affect JAK2 directly and indeed, preliminary data suggest that OdDHL does not interfere with JAK2 phosphorylation in serum-stimulated cells (LL and PES, unpublished). JAK signalling appears to extend beyond STAT activation as tyrosine residues phosphorylated by JAK2 on either ErbB2 or gp130 have been shown to serve as docking sites for Grb2 and SHP2 respectively. Both proteins, either directly or through Gab1, can transmit signals to the ERK cascade (8) and references within (28). Activation of this hypothetical pathway would be consistent with immediate early gene expression and resultant autocrine secretion, which are implicated as downstream events in the pathway blocked by OdDHL.

One possible explanation for the reciprocal effects of OdDHL on STAT activation by EGF and serum is that augmentation of the one occurs to the detriment of the other. Several models for the activation of STATs have been proposed, a common aspect of which is the recruitment of STATs to phosphorylated receptor chains from a pool of latent cytoplasmic monomers. However, recent reports indicate that a fraction of cytoplasmic STATs is not present as monomers but rather in multi-protein complexes (15). Conceivably, distinct STAT fractions are targeted by different activation mechanisms. Following growth factor stimulation, nuclear translocation of STAT3 has been shown to require receptor-mediated endocytosis, but the existence of alternative pathways has not been ruled out (1). Notably, separate nuclear import pathways have been identified for monomers and dimers of STAT1 that are predicted to be conserved among STAT proteins (13). Thus there is scope for OdDHL to act selectively during STAT activation.

An alternative possibility is that OdDHL has two molecular targets, one that is activated, possibly at low OdDHL concentrations, and another that is inactivated at higher concentrations. This model would be consistent with the observed increase in STAT3 phosphorylation in response to serum at lower OdDHL concentrations and the sharp threshold for inactivation above 50 μM seen in FIG. 15. The nature of serum as a mixed agonist means that multiple signals are likely to contribute to the net stimulation of STAT proteins. We are currently using a series of chemical analogues to test this possibility. Ultimately the identification of the molecular target(s) for OdDHL in eukaryotic cells will help to resolve these questions.

We analysed the effect of both OdDHL and OHHL on the Phosphotidylinositol-30H Kinase (P13K) survival pathway. As shown in FIG. 21, Akt phosphorylation was induced in serum-stimulated cells and blocked, as expected, by Wortmannin, an established inhibitor of P13K. OdDHL blocked phosphorylation of Akt/PKB to a similar degree as Wortmannin in BR293, MCF-7 and MDA-MB-468 cells. A titration showed it to be effective only at concentrations above 50 μM. OHHL had no effect on P13K signalling.

Materials and Methods—1

Cell Culture and Extract Preparation

Breast cancer cell lines (BR293, BT20, MCF-7, MDA-MB-231, MDA-MB-468, T47D) were maintained in Minimum Essential Medium Eagle (MEM, Sigma) supplemented with 10% foetal bovine serum (FCS), 1% MEM non-essential amino acids, 1% glutamine and 1% penicillin-streptomycin at 37° C. under 5% CO₂. These cell lines are well-known, and widely available to persons in the field. In particular, three of the lines are deposited with the American Type Culture Collection (ATTC) with the following identifying codes:

| Cell line | ATCC Number |
|---|---|
| MDA-MB-468 | HTB-132 |
| MCF7 | HTB-22 |
| MDA-MB-231 | HTB-26 |

For preparation of extracts for electrophoretic mobility shift assays (EMSAs), cells were seeded in 6-well plates (Costar) and cultured until confluent. Thereafter the cells were maintained in serum-free medium overnight before application of appropriate stimuli. For whole cell extracts, cells were lysed in TSET buffer (10 mM Tris-HCl, pH7.0, 50 mM NaCl, 1 mM EDTA, 1% Triton X-100) supplemented with 2 mM Na₃ VO₄, 5 mM Na₄P₂O₇, 5 mM NaF, 5 mM EDTA, 2 mM Benzamidine, 0.2 mM PMSF, 1 mM DTT and 1 g/ml of each leupeptin, aprotinin, pepstatin.

Extracts were cleared by centrifugation at 16,000×g for 10 minutes, snap-frozen in liquid N₂ and stored at −80° C. Nuclear extracts were prepared in high salt hypertonic buffer (20 mM HEPES pH7.9, 420 mM NaCl, 20% glycerol, 1 mM EDTA, 1 mM EGTA, 0.2% NP-40, 20 mM NaF, 1 mM Na₃ VO₄, 1 mM Na₄P₂O₇, 2 mM Benzamidine, 0.5 mM PMSF, 1 mM DTT and 1 μg/ml each of leupeptin, aprotinin and pepstatin.

For immunoprecipitation and immunoblotting experiments, cells were grown to confluence in 10 cm dishes and maintained in full medium or starved in serum-free medium overnight before the application of appropriate stimuli. Lysates were prepared in TBSN buffer (50 mM Tris-HCl pH7.5, 150 mM NaCl, 1 mM EDTA, 1% NP-40) supplemented with protease inhibitors (1 mM Na₃ VO₄, 10 mM Na₄P₂O₇, 10 mM NaF, 5 mM EGTA, 10 mM Benzamidine, 0.2 mM PMSF and 1 μg/ml each of leupeptin, aprotinin, pepstatin). Lysates were cleared by centrifugation at 16,000×g for 10 minutes and used directly for immunoprecipitations or stored at −20° C. for further use. AG490 was purchased from Sigma: PD 153035 was provided by Glaxo Wellcome and PD 168393 was purchased from Calbiochem.

Antibodies

The αSTAT1, αSTAT3, αphosphotyrosine (PY20) and αErbB1 monoclonal antibodies were purchased from Transduction Laboratories; the αErbB2, αJAK2antisera, the αErbB3 monoclonal antibody, the αphospho-STAT1 (polyclonal) and the αphospho-STAT3 (monoclonal) antibodies were purchased from Upstate Biotechnology; the rabbit polyclonal αSTAT1 and αSTAT3 antisera were made in our laboratory. The αErbB1 monoclonal antibody used for immunoprecipitations was kindly provided by Dr Lindy Durrant (University of Nottingham, UK).

Immunoprecipitation and Immunoblotting

Equal amounts of lysates were incubated with the appropriate antibody for 2 hours at 4° C.

Immune complexes were then allowed to bind to protein A-Sepharose beads for 1 hour at 4° C. and collected by centrifugation. Immunoprecipitates were washed three times in 100 mM Tris-HC1 pH 7.5, 100 nM NaCl, 1 mM EDTA, 0.1 mM PMSF, 0.5% NP-40. Thereafter, samples were taken up in sodium dodecyl sulphate (SDS) loading buffer and boiled for 5 min.

Samples were separated by electrophoresis through 6% polyacrylamide-SDS gels. Proteins were then transferred to polyvinylidene difluoride (PVDF) membranes with a semi-dry electroblotting apparatus. The membranes were incubated with appropriate primary antibodies at room temperature for 1 hour or 4° C. overnight according to suppliers' instructions, washed and stained with horseradish peroxidase-coupled secondary antibodies. The membranes were developed with an enhanced chemiluminescence kit (Amersham).

Electrophoretic Mobility Shift Assays (EMSAs)

DNA binding assays were carried out as previously described. Briefly, DNA binding by STAT proteins was analysed with a ³²P-labelled oligonucleotide duplex (M67SIE). Extracts were incubated with the DNA probe and protein-DNA complexes were separated by electrophoresis on 5% polyacrylamide gels containing 2.5% glycerol in 0.5×Tris-Borate-EDTA (TBE) buffer. After separation, the gels were fixed, dried and analysed with a phosphorimager (Fuji). For supershift analyses of STAT-DNA complexes, extracts were pre-incubated with αSTAT1 or αSTAT3 antisera at room temperature for 1 hour. Theoligonucleotide probe was then added and the EMSA was performed as described above.

Plasmids and Oligonucleotides

The expression vectors for wild type and dominant-negative STAT3 proteins (STAT3-E/V) and STAT3-Y705F) were generous gifts of Drs Curt Horvath(Mount Sinai, USA) and James E Darnell Jr (Rockefeller, USA) and have been characterised previously.

The sequences of the oligonucleotides used to generate the M67 EMSA probe, which was derived from the vSis-inducible element (SIE) of the human c-fos promoter are:

| | |
|---|---|
| Upper strand: | 5′-CTAGCATTTCCCGTAAAT |
| Lower strand: | 5′-CTAGATTTACGGGAAATG |

Cell Proliferation Assays

Equal numbers of BR293 and MDA-MB-468 cells were seeded in MEM containing 10% FCS into 10 cm dishes. Cells were allowed to grow in the presence of the JAK inhibitor AG490 (100 μM) or the ErbB1 inhibitors PD 153035 (100 nM) and PD 168393 (2 μM) for 24 or 48 hours. For the 48 time points, fresh medium containing the appropriate inhibitor was applied to the cells after 24 hours. Controls were allowed to grow for 48 hours in the absence of inhibitor. Thereafter, all the cells were washed twice with ice cold PBS, harvested and counted under a phase-contrast microscope. Values are expressed as averages+S.D. (n=3).

For the proliferation assays with wild type and dominant negative STAT3 mutants, equal numbers of MDA-MD-468 or MCF-7 cells, maintained in MEM supplemented with 10% FCS, were transfected by DNA-calcium co-precipitation with 4 μg of the corresponding expression vector or the control vector(pRc/CMV). After 96 hours, cells were harvested and processed as described above.

Materials and Methods—2

Cell Culture and Extract Preparation

Breast cancer cell lines (BR293, MCF-7 and MDA-MB-468) were maintained in Minimum Essential Medium Eagle (MEM, Sigma) supplemented with 10% foetal calf serum (FCS), 1% MEM non-essential amino acids, 1% glutamine and 1% penicillin-streptomycin at 37° C. under 5% CO₂. MCF-10F cells, one of a series of non-tumorigenic lines derived from benign breast epithelial tissue, were grown as adherent cells in a 2:1 mixture of Minimum Essential Medium Eagle and Ham's F12 medium (Sigma) supplemented with 5% horse serum, 2 mM glutamine, 10 μg ml⁻¹ insulin, 20 ng ml⁻¹ EGF, 100 ng ml⁻¹ cholera toxin, 0.5 μg ml⁻¹ hydrocortisone and 1% gentamycin. These cell lines are well-known, and widely available to persons in the field. In particular, the following lines are deposited with the American Type Culture Collection (ATTC) with the following identifying codes:

| Cell line | ATCC Number |
|---|---|
| MDA-MB-468 | HTB-132 |
| MCF7 | HTB-22 |
| MDA-MB-231 | HTB-26 |

For the preparation of nuclear extracts for electrophoretic mobility shift assays (EMSAs), cells were seeded in 10 cm dishes and cultured until confluent. Thereafter the cells were maintained in serum-free medium overnight before application of appropriate stimuli.

Nuclear extracts were prepared as described previously (11) in high salt hypertonic buffer (2 mM HEPES pH7.9, 420 mM NaCl, 20% glycerol, 1 mM EDTA, 1 mM EGTA, 20 mM NaF, 1 mM Na₃VO₄, 1 mM Na₄P₂O₇, 2 mM Benzamidine, 0.5 mM PMSF, 1 mM DTT and 1 μg/ml each of leupeptin, aprotinin and pepstatin.

For immunoprecipitation and immunoblotting experiments, cells were grown to confluence in 10 cm dishes and maintained in full medium or starved in serum-free medium overnight before the application of appropriate stimuli. Lysates were prepared in TBSN buffer (50 mM Tris-HCl pH7.5, 150 mM NaCl, 1 mM EDTA, 1% NP-40) supplemented with protease inhibitors (1 mM Na₃VO₄, 10 mM Na₄P₂O₇, 10 mM NaF, 5 mM EGTA, 10 mM Benzamidine, 0.2 mM PMSF and 1 μg/ml each of leupeptin, aprotinin and pepstatin). Lysates were cleared by centrifugation at 16,000×g for 10 minutes and used directly for immunoprecipitations or stored at −20° C. for further use. AG490 was purchased from Sigma; PD153035 was provided by Glaxo-Smith-Kline and PD168393 was purchased from Calbiochem.

Plasmids and Oligonucleotides

The luciferase reporter construct pSIE2-luc contains 2 copies of the M67 site inserted upstream of the adenovirus 2 E4 basal promoter.

The sequences of the oligonucleotides used to generate the M67 EMSA probe, which was derived from the vSis-inducible element (SIE) of the human c-fos promoter, are:

| | |
|---|---|
| Upper strand: | 5′-CTAGCATTTCCCGTAAAT |
| Lower strand: | 5′-CTAGATTTACGGGAAATG |

Antibodies

The anti-STAT3, anti-phosphotyrosine (PY20) and anti-ErbB1 monoclonal antibodies were purchased from Transduction Laboratories; the anti-phospho-STAT3 (monoclonal) antibody was purchased from Upstate Biotechnology; the rabbit polyclonal anti-STAT3 antisera was made in our laboratory (11); the anti-ErbB1 monoclonal antibody used for immunoprecipitations was kindly provided by Dr. Lindy Durrant (University of Nottingham, UK); the anti-PARP antibody was purchased from New England Biolabs.

Immunoprecipitation and Immunoblotting

Equal amounts of lysates were incubated with the appropriate antibody for 2 hours at 4° C. Immune complexes were then allowed to bind to protein A-Sepharose beads for 1 hour at 4° C. and collected by centrifugation. Immunoprecipitates were washed three times in 10 mM Tris-HCl pH 7.5, 100 mM NaCl, 1 mM EDTA, 0.1 mM PMSF, 0.5% NP-40. Thereafter, samples were taken up in sodium dodecyl sulphate (SDS) loading buffer and boiled for 5 min.

Samples were separated by electrophoresis through 6% polyacrylamide-SDS gels. Proteins were then transferred to polyvinylidene difluoride (PVDF) membranes with a semi-dry electroblotting apparatus. The membranes were incubated with appropriate primary antibodies at room temperature for 1 hour or 4° C. overnight according to suppliers' instructions, washed and stained with horseradish peroxidase-coupled secondary antibodies. The membranes were developed with an enhanced chemiluminescence kit (Amersham).

Electrophoretic Mobility Shift Assays (EMSAs)

DNA binding assays were carried out as previously described (11). Briefly, DNA binding by STAT proteins was analysed with a ³²P-labelled oligonucleotide duplex (M67SIE). Extracts were incubated with the DNA probe and protein-DNA complexes were separated by electrophoresis on 5% polyacrylamide gels containing 2.5% glycerol in 0.5×Tris-Borate-EDTA (TBE) buffer. After separation, the gels were fixed, dried and analysed with a phosphorimager (Fuji). For supershift analyses of STAT-DNA complexes, extracts were pre-incubated with anti-STAT3 antiserum at room temperature for 1 hour. The oligonucleotide probe was then added and the EMSA was performed as described above.

Cell Proliferation Assays

Equal numbers of BR293, MCF-10F, MCF-7, MDA-MB-468, HKEK293 and COS1 cells were seeded in the appropriate growth medium into 10 cm dishes. Cells were allowed to grow in the presence of various concentrations of OdDHL or OHHL for 24 or 48 hours. For the 48-hour time points, fresh medium containing the appropriate inhibitor was applied to the cells after 24 hours. Controls were allowed to grow for 48 hours in the absence of inhibitor. Thereafter, all the cells were washed twice with ice cold PBS, harvested and counted under a phase-contrast microscope. Values are expressed as averages ±S.D. (n=3).

For [³H]-thymidine incorporation assays, BR293 cells were seeded into 96 well plates (2×10⁴/well) in DME supplemented with 10% FCS and grown over night. The medium was then replaced with serum-free medium. Twenty-four hours later, the designated medium was added and after 18 h, 0.5 μCi of (methyl)-[³H]-thymidine (20 Ci mmol⁻¹, Amersham Corp.) was added to each well for an additional 18 h. The cells were then harvested with trypsin/EDTA and transferred to a cellulose-coated plate and washed. Incorporated radioactivity was measured with a microplate scintillation counter. Values are expressed as averages +/−SD (n=4).

### REFERENCES

[1] Bild, A. S., J. Turkson, and R. Jove. 2002. Cytoplasmic transport of STAT3 by receptor-mediated endocytosis. EMBO J. 21:3255-3263.

[2] Bowman, T., R. Garcia, J. Turkson, and R. Jove. 2000. STATs in oncogenesis. Oncogene 19:2474-2488.

[3] Bromberg, J., and J. E. J. Darnell. 2000. The role of STATs in transcriptional control and their impact on cellular function. Oncogene 19:2468-2473.

[4] Bromberg, J. F., M. H. Wrzeszczynska, G. Devgan, Y. Zhao, R. G. Pestell, C. Albanese, and J. E. J. Darnell. 1999. Stat3 as an Oncogene. Cell 98:295-303.

[5] Burke, W. M., X. Jin, H. J. Lin, M. Huang, R. Liu, R. K. Reynolds, and J. Lin. 2001. Inhibition of constitutively active STAT3 suppresses growth of human ovarian and breast cancer cells. Oncogene 20:7925-7934.

[6] Catlett-Falcone, R., T. H. Landowski, M. M. Oshiro, J. Turkson, A. Levitzki, R. Savino, G. Ciliberto, L. Moscinski, J. L. Femandez-Luna, G. Nunez, W. S. Dalton, and J. R. 1999. Constitutive activation of Stat3 signaling confers resistance to apoptosis in human U266 myeloma cells. Immunity 10:105-15.

[7] Garcia, R., T. L. Bowman, G. Niu, H. Yu, S. Minton, C. A. Muro-Cacho, C. E. Cox, R. Falcone, R. Fairclough, S. Parsons, A. Laudano, A. Gazit, A. Levitzki, A. Kraker, and R. Jove. 2001. Constitutive activation of Stat3 by the Src and JAK tyrosine kinases participates in growth regulation of human breast carcinoma cells. Oncogene 20:2499-2513.

[8] Hirano, T., K. Ishihara, and M. Hibi. 2000. Roles of STAT3 in mediating the cell growth, differentiation and survival signals relayed through the IL-6 family of cytokine receptors. Oncogene 19:2548-2556.

[9] Kölch, W. 2000. Meaningful relationships: the regulation of the Ras/Raf/MEK/ERK pathway by protein interactions. Biochem. J. 351:289-305.

[10] Kotenko, S. V., and S. Pestka. 2000. Jak-Stat signal transduction pathway through the eyes of cytokine class II receptor complexes. Oncogene 19:2557-2565.

[11] Li, L., and P. E. Shaw. 2002. Autocrine-Mediated Activation of STAT3 Correlates With Cell Proliferation in Breast Carcinoma Lines. J. Biol. Chem. 277:17397-17405.

[12] Lin, T. S., S. Mahajan, and D. A. Frank. 2000. STAT signalling in the pathogenesis and treatment of leukaemias. Oncogene 19:2496-2504.

[13] Meyer, T., A. Begitt, I. Lodige, M. van Rossum, and V. U. 2002. Constitutive and IFN-g-induced nuclear import of STAT1 proceed through independent pathways. EMBO J. 21:344-354.

[14] Mochizuki, T., C. Kitanaka, K. Noguchi, T. Muramatsu, A. Asai, and Y. Kuchino. 1999. Physical and functional interactions between Pim-1 kinase and Cdc25A phosphatase. Implications for the Pim-1-mediated activation of the c-Myc signaling pathway. J. Biol. Chem. 274:18659-66.

[15] Ndubuisi, M. I., G. G. Guo, V. A. Fried, E. J.D., and S. P.B. 1999. Cellular physiology of STAT3: Where's the cytoplasmic monomer? J. Biol. Chem. 274:25499-25509.

[16] Pesci, E. C., J. P. Pearson, P. C. Seed, and B. H. Iglewski. 1997. Regulation of las nad rh1 quorum sensing in Pseudomonas aeruginosa. J. Bacteriol. 179:3127-3132.

[17] Reddy, E. P., A. Korapati, P. Chaturvedi, and S. Rane. 2000. IL-3 signalling and the role of Src kinases, JAKs and STATs: a covert liaison unveiled. Oncogene 19:2532-2547. [18] Robinson, M. J., and M. H. Cobb. 1997. Mitogen-Activated Protein Kinase Pathways. Curr. Opin. Cell Biol. 9:180-186.

[19] Sano, M., K. Fukuda, H. Kodama, T. Takahashi, T. Kato, D. Hakuno, T. Sato, T. Manabe, S. Tahara, and S. Ogawa. 2000. Autocrine/Paracrine Secretion of IL-6 Family Cytokines Causes Angiotensin 1′-Induced Delayed STAT3 Activation. Biochem. Biophys. Res. Comm. 269:798-802.

[20] Seidel, H. M., P. Lamb, and J. Rosen. 2000. Pharmaceutical intervention in the JAK/STAT signalling pathway. Oncogene 19:2645-2656.

[21] Shirogane, T., T. Fukada, J. M. Muller, D. T. Shima, M. Hibi, and T. Hirano. 1999. Synergistic roles for Pim-1 and c-Myc in STAT3-mediated cell cycle progression and antiapoptosis. Immunity 11:709-19.

[22] Simon, A. R., H. G. Vikis, S. Stewart, B. A. Fanburg, B. H. Cochran, and K.-L. Guan. 2000. Regulation of STAT3 by Direct Binding to the Rac1 GTPase. Science 290:144-147.

[23] Smith, R. S., S. G. Harris, R. Phipps, and B. Iglewski. 2002. The Pseudomonas aeruginosa quorum-sensing molecule N-(3-oxododecanoyl) homoserine lactone contributes to virulence and induces inflammation in vivo. J. Bacteriol. 184:1132-1139.

[24] Smithgall, T. E., S. D. Briggs, S. Schreiner, E. C. Lemer, H. Cheng, and M. B. Wilson. 2000. Control of myeloid differentiation and survival by Stats. Oncogene 19:2612-2618.

[25] Telford, G., D. Wheeler, P. Williams, P. T. Tomkins, P. Appleby, H. Sewell, G. S. A. B. Stewart, B. W. Bycroft, and D. I. Pritchard. 1998. The Pseudomonas aeruginosa quorum sensing signal molecule, N-(3-oxododecanoyl)-L-homoserine lactone, has immunomodulatory activity. Infect. Immun. 66:36-42.

[26] Turkson, J., and R. Jove. 2000. STAT proteins: novel molecular targets for cancer drug discovery. Oncogene 19:6613-6626.

[27] Winson, M. K., M. Camara, A. Latifi, M. Foglino, S. R. Chhabra, M. Daykin, M. Bally, V. Chapon, G. P. Salmond, B. W. Bycroft, A. Lazdunski, G. S. Stewart, and P. Williams. 1995. Multiple N-acyl-L-homoserine lactone signal molecules regulate production of virulence determinants and secondary metabolites in Pseudomonas aeruginosa. Proc. Natl. Acad. Sci. USA 92:9427-31.

[28] Yamauchi, T., N. Yamauchi, K. Ueki, T. Sugiyama, H. Waki, H. Miki, K. Tobe, S. Matsuda, T. Tsushima, T. Yamamoto, T. Fujita, Y. Taketani, M. Fukayama, S. Kimura, Y. Yazaki, R. Nagai, and T. Kadowaki. 2000. Constitutive tyrosine phosphorylation of ErbB-2 via JAK2 by autocrine secretion of prolactin in human breast cancer. J. Biol. Chem. 275:33937-44.

[29] Wagner, B. J., T. E. Hayes, C. J. Hoban, and B. H. Cochran. 1990. The SIF binding element confers sis/PDGF inducibility onto the c-fos promoter. EMBO J. 9:4477-4484.

1-17. (Cancelled)

18. The modulation of an autocrine/paracrine signalling pathway which activates STAT wherein the pathway requires JAK activity and does not require Erb1 activity and is not induced by EGF, to alter the amount of activated STAT.

19. The modulation of a process wherein STAT dimers accumulate in the cytoplasm wherein the process does not require ErbB1 activity or JAK activity, to alter the amount of activated STAT.

20. The use of a compound selected from: JAK, ErbB1, EGF, ErbB1 inhibitors, EGF inhibitors, STAT inhibitors, interleukin-13 (IL-13), IL-13E13K (IL-13 in which the Glu at position 13 is substituted by a Lys residue), sulpher methoxyzol, ubiquitin E3 ligase, serine phosphatase, tyrosine phosphotase, SOCs, Pias proteins (protein inhibitors of activated STAT), STAT1 inhibitors, STAT2 inhibitors, STAT3 inhibitors, STAT4 inhibitors, STAT5A inhibitors, STAT5B inhibitors, STAT6 inhibitors, JAK inhibitors, AG 490, α-amanitin, transcription inhibitors, quorum sensing molecules, N-acyl homoserine lactones, N-(3-oxododecanoyl)-L-homoserine lactone, oxygen radical scavengers, N-acetyl Cysteine (NAC), diphenylene iodonium chloride (DPI), inhibitors of COX1, inhibitors of COX2, aspirin, ketorolac, indomethacin, or panCOX inhibitors to modulate STAT activity for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukemia (ALL), chronic lymphocytic leukaemia (CLL), acute myelogenous leukemia (AML), chronic myelogenous leukaemia (CML), megakaryoticleukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

21. The use of a compound of the formula I: 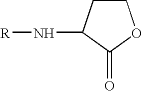
in which R is an acyl group of the formula II: 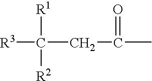
wherein one of R¹ and R² is H and the other is selected from OR⁴, SR⁴ and NHR4 wherein R⁴ is H or 1-6C alkyl, or R¹ and R² together with the carbon atom to which they are joined form a keto group and R³ is a straight or branched chain saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of the R⁵ and R⁶ is selected from H and 1-6C alkyl or R⁵ and R⁶ together with the N atom form a morpholino or piperazino group or any enantiomer thereofwith the proviso that R is not a 3-oxododecanoyl group to modulate STAT activity for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

22. The use claimed in claim 21 wherein the R group is selected from 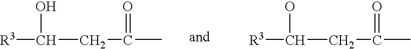
wherein R³ is as defined in claim 21.

23. The use claimed in claim 21 wherein the group R³ is an 8-11C straight or branched chain alkyl group optionally substituted by a substituent selected from bromo, carboxy and methoxycarbonyl.

24. The use claimed in claim 22 wherein the group R³ is an 8-11C straight or branched chain alkyl group optionally substituted by a substituent selected from bromo, carboxy and methoxycarbonyl.

25. The use claimed in claim 21 wherein the R³ group is such that the group R in formula I is selected from:
3-oxoundecanoyl;
11-bromo-3-oxoundecanoyl;
10-methyl-3-oxoundecanoyl;
6-methyl-3-oxoundecanoyl;
3-hydroxydodecanoyl;
12-bromo-3-oxododecanoyl;
3-oxotridecanoyl;
13-bromo-3-oxotridecanoyl;
3-hydroxytetradecanoyl;
3-oxotetradecanoyl;
14-bromo-3-oxotradecanoyl; and
13-methoxycarbonyl-3-oxotridecanoyl.

26. The use claimed in claim 21 wherein the R³ is an 8-11 straight or branched chain alkenyl group optionally substituted by a substituent selected from bromo, carboxy and methoxycarbonyl.

27. The use claimed in claim 21 wherein the R³ group is such that the group R in formula I is selected from;
3-oxo-12-tridecenoyl;
3-oxo-7-tridecenoyl;
3-hydroxy-7-tetradecenoyl;
3-oxo-9-tetradecenoyl;
3-hydroxy-9-tetradecenoyl;
3-oxo-10-tetradecenoyl;
3-hydroxy-10-tetradecenoyl;
3-oxo-11-tetradecenoyl;
3-hydroxy-1-tetradecenoyl;
3-oxo-13-tetradecenoyl; and
3-hydroxy-13-tetradecenoyl.

28. The use of JAK, ErbB1, EGF, ErbB1 inhibitors, EGF inhibitors, STAT inhibitors, interleukin-13 (IL-13), IL-13E13K (IL-13 in which the Glu at position 13 is substituted by a Lys residue), sulpher methoxyzol, ubiquitin E3 ligase, serine phosphatase, tyrosine phosphotase, SOCs, Pias proteins (protein inhibitors of activated STAT), STAT1 inhibitors, STAT2 inhibitors, STAT3 inhibitors, STAT4 inhibitors, STAT5A inhibitors, STAT5B inhibitors, STAT6 inhibitors, JAK inhibitors, AG 490, α-amanitin, transcription inhibitors, quorum sensing molecules, N-acyl homoserine lactones, N-(3-oxododecanoyl)-L-homoserine lactone, oxygen radical scavengers, N-acetyl Cysteine (NAC), diphenyleneiodonium chloride (DPI), inhibitors of COX1, inhibitors of COX2, aspirin, ketorolac, indomethacin, or panCOX inhibitors for the preparation of a medicament for the treatment of cancer, breast cancer, multiple myeloma, head and neck cancers, leukaemia, HTLV-1-dependent leukemia, large granular lymphocte (LGL) leukaemia, erythroleukemia, acute lymphocytic leukaemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocarcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immune deficiency or immune disorders.

29. The use of a compound of the formula I: 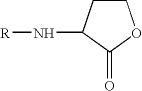
in which R is an acyl group of the formula II: 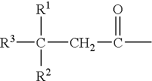
wherein one of R¹ and R² is H and the other is selected from OR⁴, SR⁴ and NHR⁴ wherein R⁴ is H or 1-6C alkyl, or R¹ and R² together with the carbon atom to which they are joined form a keto group and R³ is a straight or branched chain saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of the R⁵ and R⁶ is selected from H and 1-6C alkyl or R⁵ and R⁶ together with the N atom form a morpholino or piperazino group or any enantiomer thereofwith the proviso that R is not a 3-oxododecanoyl group for the preparation of a medicament for the treatment of treatment of cancer, breast cancer, multiple myeloma, head and neck cancers, leukaemia, HTLV-1-dependent leukemia, large granular, lymphocyte (LGL) leukaemia (ALL), chronic lymphocytic leukaemia (CLL), acute myelogenous leukemia (AML), chronic myelogenous leukaemia (CML), megakaryotic leukaemia, lung cancer, renal cell carcinoma, prostrate carcinoma, melanoma, ovarian carcinoma, pancreatic adenocurcinoma, lymphoma, EBV-related lymphoma, Burkitt's lymphoma mycosis fungoides lymphoma, HSV saimiri-dependent (T cell) lymphoma, cutaneous T cell lymphoma, obesity, lipid metabolism disorders, immune disease, immunedeficiency or immune disorders.

30. The use claimed in claim 29 wherein the R group is selected from 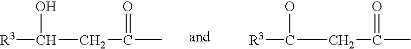
wherein R³ is as defined in claim 29.

31. The use in claim 29 wherein the group R³ is an 8-11C straight or branched chain alkyl group optionally substituted by a substituent selected from bromo, carboxy and methoxycarbonyl.

32. The use claimed in claim 29 wherein the R³ group is such that the group R in formula I is selected from;
3-oxoundecanoyl;
11-bromo-3-oxoundecanoyl;
10-methyl-3-oxoundecanoyl;
6-methyl-3-oxoundecanoyl;
3-hydroxydodecanoyl;
12-bromo-3-oxododecanoyl;
3-oxotridecanoyl;
13-bromo-3-oxotridecanoyl;
3-hydroxytetradecanoyl;
3-oxotetradecanoyl;
14-bromo-3-oxotradecanoyl; and
13-methoxycarbonyl-3-oxotridecanoyl.

33. The use claimed in claim 29 wherein the R³ is an 8-11 straight or branched chain alkenyl group optionally substituted by a substituent selected from bromo, carboxy and methoxycarbonyl.

34. The use claimed in claim 29 wherein the R³ group is such that the group R in formula I is selected from;
3-oxo-12-tridecenoyl;
3-oxo-7-tridecenoyl;
3-hydroxy-7-tetradecenoyl;
3-oxo-9-tetradecenoyl;
3-hydroxy-9-tetradecenoyl;
3-oxo-10-tetradecenoyl;
3-hydroxy-10-tetradecenoyl;
3-oxo-11-tetradecenoyl;
3-hydroxy-11-tetradecenoyl;
3-oxo-13-tetradecenoyl; and
3-hydroxy-13-tetradecenoyl.
